SEQUENCE LISTING (45 sequences)

SEQ ID NO: 1 - DNA, 20 bp, Artificial Sequence
  Synthetic B2M crRNA targeting sequence B2M exon 1
gagtagcgcgagcacagcta

SEQ ID NO: 2 - DNA, 20 bp, Artificial Sequence
  Synthetic B2M crRNA targeting sequence B2M exon 2
agtcacatggttcacacggc

SEQ ID NO: 3 - DNA, 20 bp, Artificial Sequence
  Synthetic CIITA crRNA targeting sequence CIITA exon 2
catcgctgttaagaagctcc

SEQ ID NO: 4 - DNA, 20 bp, Artificial Sequence
  Synthetic CIITA crRNA targeting sequence CIITA exon 3
gatattggcataagcctccc

SEQ ID NO: 5 - DNA, 20 bp, Artificial Sequence
  Synthetic Primer B2M Exon 1 Forward
atataagtggaggcgtcgcg

SEQ ID NO: 6 - DNA, 20 bp, Artificial Sequence
  Synthetic Primer B2M Exon 1 Reverse
tggagagactcacgctggat

SEQ ID NO: 7 - DNA, 21 bp, Artificial Sequence
  Synthetic Primer B2M Exon 2 Forward
tgtctttcagcaaggactggt

SEQ ID NO: 8 - DNA, 22 bp, Artificial Sequence
  Synthetic Primer B2M Exon 2 Reverse
accccacttaactatcttgggc

SEQ ID NO: 9 - DNA, 20 bp, Artificial Sequence
  Synthetic Primer CIITA Exon 2 Forward
ctgcctctttccaacaccct

SEQ ID NO: 10 - DNA, 20 bp, Artificial Sequence
  Synthetic Primer CIITA Exon 2 Reverse
cttctccagccaggtccatc

SEQ ID NO: 11 - DNA, 20 bp, Artificial Sequence
  Synthetic Primer CIITA Exon 3 Forward
tttcagcaggctgttgtgtg

SEQ ID NO: 12 - DNA, 20 bp, Artificial Sequence
  Synthetic Primer CIITA Exon 3 Reverse
gcagcaaagaactcttgccc

SEQ ID NO: 13 - DNA, 20 bp, Artificial Sequence
  Synthetic Off-target site of B2M exon 2 HDHD1P2
tgagagtaccaggtgtgacg

SEQ ID NO: 14 - DNA, 34 bp, Artificial Sequence
  Synthetic Synthetic Primer HDHD1P2_F
tcgtcggcagcgtcgtgcagtctgggatttggga

SEQ ID NO: 15 - DNA, 35 bp, Artificial Sequence
  Synthetic Primer HDHD1P2_R
gagggccgtctcgtgggctcggtatgagtgagagg

SEQ ID NO: 16 - DNA, 23 bp, Artificial Sequence
  Synthetic Off-target coding site of CIITA exon 2 JMJD4
catcactgctaggaagcttcagg

SEQ ID NO: 17 - DNA, 34 bp, Artificial Sequence
  Synthetic Primer JMJD4_F
tcgtcggcagcgtcatcaaaggctgcctgttcga

SEQ ID NO: 18 - DNA, 35 bp, Artificial Sequence
  Synthetic Primer JMJD4_R
gtctcgtgggctcggtgctcgggcatcaactttga

SEQ ID NO: 19 - DNA, 23 bp, Artificial Sequence
  Synthetic Off-target coding site of CIITA exon 3 OSTF1
gatatctgcataacccttccagg

SEQ ID NO: 20 - DNA, 35 bp, Artificial Sequence
  Synthetic Primer OSTF1_F
tcgtcggcagcgtcgggagatacagctttgcatgc

SEQ ID NO: 21 - DNA, 35 bp, Artificial Sequence
  Synthetic Primer OSTF1_R
aagaccagtctcgtgggctcggttcagggcaagca

SEQ ID NO: 22 - DNA, 20 bp, Artificial Sequence
  Synthetic Primer sense
ccacatcgctcagacaccat

SEQ ID NO: 23 - DNA, 17 bp, Artificial Sequence
  Synthetic Primer antisense
tgaccaggcgcccaata

SEQ ID NO: 24 - DNA, 17 bp, Artificial Sequence
  Synthetic Probe
agtcaacggatttggtc

SEQ ID NO: 25 - DNA, 22 bp, Artificial Sequence
  Synthetic Primer sense
tgtccacgtgttgagatcattg

SEQ ID NO: 26 - DNA, 20 bp, Artificial Sequence
  Synthetic Primer antisense
ggccttcgattctggattca

SEQ ID NO: 27 - DNA, 24 bp, Artificial Sequence
  Synthetic Probe
tacaatgaaaaagaagggtgagaa

SEQ ID NO: 28 - DNA, 18 bp, Artificial Sequence
  Synthetic B2M Exon 1 gRNA
atcgacgagcgcgatgag

SEQ ID NO: 29 - DNA, 30 bp, Artificial Sequence
  Synthetic B2M Exon 1
tccgtggccttagctgtgctcgcgctactc

SEQ ID NO: 30 - DNA, 28 bp, Artificial Sequence
  Synthetic B2M Exon 1
tccgtggcctgctgtgctcgcgctactc

SEQ ID NO: 31 - DNA, 29 bp, Artificial Sequence
  Synthetic B2M Exon 1
tccgtggcctagctgtgctcgcgctactc

SEQ ID NO: 32 - DNA, 19 bp, Artificial Sequence
  Synthetic B2M Exon 2 gRNA
cacacttggtacactgact

SEQ ID NO: 33 - DNA, 33 bp, Artificial Sequence
  Synthetic B2M Exon 2
tgagtatgcctgccgtgtgaaccatgtgactga

SEQ ID NO: 34 - DNA, 22 bp, Artificial Sequence
  Synthetic B2M Exon 2
tgagtgtgaaccatgtgactga

SEQ ID NO: 35 - DNA, 22 bp, Artificial Sequence
  Synthetic B2M Exon 2
tgagtatgcctgccgtgactga

SEQ ID NO: 36 - DNA, 26 bp, Artificial Sequence
  Synthetic B2M Exon 2
tgagtatgcctgaaccatgtgactga

SEQ ID NO: 37 - DNA, 20 bp, Artificial Sequence
  Synthetic CIITA EXON 2 gRNA
cctcgaagaattgtcgctac

SEQ ID NO: 38 - DNA, 27 bp, Artificial Sequence
  Synthetic CIITA EXON 2
gctacctggagcttcttaacagcgatg

SEQ ID NO: 39 - DNA, 17 bp, Artificial Sequence
  Synthetic CIITA EXON 2
gcttcttaacagcgatg

SEQ ID NO: 40 - DNA, 26 bp, Artificial Sequence
  Synthetic CIITA EXON 2
gctacctggacttcttaacagcgatg

SEQ ID NO: 41 - DNA, 20 bp, Artificial Sequence
  Synthetic CIITA EXON 3 gRNA
ccctccgaatacggttatag

SEQ ID NO: 42 - DNA, 27 bp, Artificial Sequence
  Synthetic CIITA EXON 3
gagaccagggaggcttatgccaatatc

SEQ ID NO: 43 - DNA, 24 bp, Artificial Sequence
  Synthetic CIITA EXON 3
gagaccagggcttatgccaatatc

SEQ ID NO: 44 - DNA, 25 bp, Artificial Sequence
  Synthetic CIITA EXON 3
gagaccaggggcttatgccaatatc

SEQ ID NO: 45 - DNA, 28 bp, Artificial Sequence
  Synthetic CIITA EXON 3
gagaccagggaaggcttatgccaatatc

(19) United States
(12) Patent Application Publication
(10) Pub. No.: US 2024/0255520 A1
(43) Pub. Date: Aug. 1, 2024

(54) HLA-DELETED GLOMERULAR ENDOTHELIAL CELLS AND DIAGNOSTIC METHOD USING THEREOF

(71) Applicants: INSTITUT NATIONAL DE LA SANTE ET DE LA RECHERCHE MEDICALE, Paris (FR); UNIVERSITÉ PARIS CITÉ, Paris (FR); CENTRE NATIONAL DE LA RECHERCHE SCIENTIFIQUE (CNRS), Paris (FR); ASSISTANCE PUBLIQUE-HÔPITAUX DE PARIS, Paris (FR)

(72) Inventors: Dany ANGLICHEAU, Paris (FR); Baptiste LAMARTHEE, Leuven (BE)

(21) Appl. No.: 18/290,096

(22) PCT Filed: May 11, 2022
(86) PCT No.: PCT/EP2022/062769

§ 371 Date: Nov. 9, 2023

(30) Foreign Application Priority Data
May 12, 2021 (EP) 21305632.8
Jul. 9, 2021 (EP) 21305960.3

(51) Int. Cl. G01N 33/68; C12N 5/071; G01N 33/50

(52) U.S. Cl. CPC G01N 33/6854; C12N 5/069; G01N 33/5005; C12N 2503/00; C12N 2510/00; G01N 2800/50

(57) ABSTRACT

The invention relates to an engineered glomerular endothelial cell comprising a reduction in expression of a human leukocyte antigen and its use in an in vitro method for determining the likelihood of occurrence of a non-HLA antibody mediated rejection against a renal allograft in an individual in need thereof. The inventors have observed that by suppressing expression of HLA class I and II molecules it was possible to obtain a cell able to bind non-HLA antibodies. The in vitro diagnostic method has been validated on a cohort of patients.

### TECHNICAL FIELD

The present invention relates to the field of organ transplant and the issues associated with transplant rejection. In particular, the present invention relates to in vitro methods for determining the likelihood of occurrence of a non-HLA antibody mediated rejection against a renal allograft and to engineered glomerular endothelial cells comprising a reduction in expression of a human leukocyte antigen (HLA) useable in the methods.

### TECHNICAL BACKGROUND

Approximately 140,000 solid-organ transplantations are performed each year worldwide (http://www.transplant-observatory.org), and two-thirds of these procedures are kidney transplantations. Despite the development of potent immunosuppressive regimens, immune-mediated allograft injury remains a significant hurdle to the long-term acceptance of solid-organ transplants, and after kidney transplantation, alloimmune injuries remain major determinants of early and late allograft dysfunctions and contribute to allograft failures, with subsequent re-initiation of dialysis and increased morbidity, mortality and cost (Nankivell, B. J. & Kuypers, D. R. The Lancet (2011). doi:10.1016/S0140-6736(11)60699-5).

Transplant rejection occurs when transplanted tissue is rejected by the recipient's immune system. It is an adaptive immune response via cellular immunity (mediated by cytotoxic T cells inducing apoptosis of target cells) as well as humoral immunity (mediated by activated B cells secreting antibody molecules) or antibody-mediated rejection (ABMR).

The description of close associations between circulating donor-specific anti-human leucocyte antigen antibodies (HLA-DSAs) and microvascular inflammation (MVI) in the kidney allograft progressively led to standardized diagnostic criteria for ABMR in the kidney allograft, which were progressively adopted and modified for other solid-organ transplantations (Loupy, A. & Lefaucheur, New England Journal of Medicine (2018). doi:10.1056/NEJMra1802677).

In kidney transplantation, the current ABMR classification requires the association of histological lesions suggestive of acute tissue injury, evidence of current or recent antibody interaction with the vascular endothelium, and serologic evidence of circulating donor-specific antibodies (DSAs). Many studies have described the main role of HLA-DSAs in the occurrence of ABMR. Nevertheless, the existence of ABMR-like histological lesions in the absence of HLA-DSAs may suggest alternative mechanisms, including the presence and deleterious impact of non-HLA antibodies (Abs). Such a non-HLA antibody-mediated acute rejection may be termed AMVR (for “Acute MicroVascular Rejection”).

Further identification of the mechanisms underlying ABMR-like histological lesions in the absence of HLA-DSAs faces several unresolved hurdles. First, a dedicated clinicopathological description of ABMR-like histological lesions in the absence of HLA-DSAs is needed to better define this particular phenotype, which is called ABMR histology (ABMRh). It has been demonstrated that 40-60% of cases with ABMRh have no circulating HLA-DSAs (DSAnegABMRh) (Senev, A. et al. Am. J. Transplant. 19, 763-780 (2019); Koenig, A. et al. Nat. Commun. 10, 5350 (2019); Bestard, O. & Grinyó, J. American Journal of Transplantation 19, 952-953 (2019).)

Second, the firm demonstration that ABMRh is due to Ab-mediated injury is hampered by the facts that many non-HLA Abs may be involved, and no single test is able to identify them. Indeed, in recent years, many targets other than HLA antigens, including angiotensin type I receptor (AT1R), endothelin-1 type A receptor (ETAR), LG3, vimentin, FLT3, ICAM4, endoglin and agrin, have been proposed.

A previous study identified kidney transplant recipients (KTRs) who experienced severe ABMRh in the first 3 months after transplantation in the absence of HLA-DSAs. In that study, assessment of previously identified non-HLA Abs failed to differentiate ABMRh patients from stable patients. However, it was demonstrated that at the time of transplantation, patients carried preformed IgG Abs specifically targeting a conditionally immortalized human glomerular endothelial cell line (CiGEnC) (Delville, M. et al. J. Am. Soc. Nephrol. 30, 692-709 (2019)).

These results suggested that in vitro cell-based assays are needed to assess the presence of non-HLA Abs with potential deleterious effects after transplantation.

WO2020144366A1 describes in vitro methods and kits using endothelial cells or conditionally immortalized human glomerular endothelial cells (CiGEnC) for determining the likelihood of occurrence of an acute microvascular rejection (AMVR) against a renal allograft in an individual.

Given that endothelial cells and CiGEnC express both class I and class II HLA molecules, the use of these cells for a cell-based assay was limited to patients with no circulating anti-HLA Abs.

Merola et al., (JCI Insight. 2019; 4(20):e129739.) describe the CRISPR/Cas9-mediated dual ablation of β2-microglobulin and class II transactivator (CIITA) in human endothelial colony-forming cells (HECFC)-derived endothelial cells (ECs) and elimination of both class I and II MHC expression. However, the cell used in this paper is not a glomerular endothelial cell.

There remains a need for improving diagnosis of a non-HLA antibody mediated rejection in transplanted patients, including in recipients of a renal allograft.

In particular, there remains a need for the provision of tools and methods allowing the prediction or the diagnosis of an antibody-mediated acute allograft rejection to which contribute anti-endothelial cells antibodies (AECAs) directed to non-HLA antigens.

There also remains a need to develop tools and methods for determining the likelihood of occurrence of a non-HLA antibody mediated rejection (or AMVR) against a renal allograft in an individual before or shortly after transplantation in order to provide said individual with a treatment which is specifically adapted and thus avoiding a graft rejection altogether. After transplant, there is also a need to develop a method for detecting the presence of circulating non-HLA anti-endothelial cells antibodies as a companion test for diagnosing an acute rejection due to non-HLA anti-endothelial cells antibodies (or AMVR).

There is a need to have a specific cellular tool allowing to detect specifically non-HLA antibodies in an individual in need thereof.

There is a need to have a specific cellular tool allowing to detect specifically non-HLA antibodies which may be easily to implement. Such tool should also ensure sensitivity and reproducibility.

There is also a need to have a diagnostic method allowing to discriminate, within patients to be transplanted with a kidney between, the patients susceptible and the patients non-susceptible to undergo antibody-mediated rejection mediated by non-HLA antibodies (or AMVR).

There is also a need to have a diagnostic tool for use in method allowing to discriminate, within patients to be transplanted with a kidney between, the patients susceptible and the patients non-susceptible to undergo antibody-mediated rejection mediated by non-HLA antibodies (or AMVR).

There is a need to have a diagnostic method implementing a specific reference value allowing to discriminate with a good sensitivity and efficacy between patients susceptible and patients non-susceptible to undergo antibody-mediated rejection mediated by non-HLA antibodies.

There is a need to have a diagnostic and treatment method allowing to monitor kidney transplantation recipients and detect a likelihood of occurrence of a non-HLA antibody mediated rejection against a renal allograft and further provide appropriate treatment to the patient.

There is a need to have a biomarker allowing for determining the likelihood of occurrence of a non-HLA antibody mediated rejection against a renal allograft which may be easy to implement.

There is also a need to have a reference value usable in diagnostic method for determining the likelihood of occurrence of a non-HLA antibody mediated rejection against a renal allograft which may be easy to implement, with good reliability and sensibility.

There is a need to have a non-HLA antibody detection cell-based assay which can be used as a companion test for kidney transplantation.

There is a need to have a non-HLA antibody detection cell-based assay which can be used for determining the likelihood of occurrence of a non-HLA antibody mediated rejection pre- and post-transplantation kidney transplant recipients.

There is a need to have a reference threshold based on a non-HLA antibody quantification allowing to discriminate within patients having received or intended to receive a kidney transplant between patients susceptible to undergo a non-HLA antibody mediated rejection and patients not susceptible to undergo a non-HLA antibody mediated rejection. The present invention has for purpose to satisfy all or part of those needs.

### SUMMARY

According to one of its objects, the present invention relates to an in vitro method for determining the likelihood of occurrence of a non-HLA antibody mediated rejection against a renal allograft in an individual in need thereof, the method comprising at least the steps of:
a) incubating at least one engineered human glomerular endothelial cell comprising a reduction in expression of a human leukocyte antigen (HLA) with a blood sample of said individual, said blood sample being presumed to contain non-HLA antibodies,
b) obtaining a quantification of a predetermined signal of non-HLA antibodies bound to said engineered human glomerular endothelial cell,
c) comparing the quantification obtained at step b) with a predetermined reference value, wherein the predetermined reference value is a ratio of a first quantification of a predetermined signal obtained from antibodies bound to an engineered glomerular endothelial cell comprising a reduction in expression of a human leukocyte antigen (HLA), said antibodies being from a blood sample of an individual known to contain non-HLA antibodies, over a second quantification of a predetermined signal obtained from an engineered glomerular endothelial cell comprising a reduction in expression of a human leukocyte antigen (HLA), in absence of non-HLA antibodies, and
d) wherein a quantification obtained at step b) greater than the predetermined reference value indicates that said individual is at risk of a non-HLA antibody mediated rejection against a renal allograft.

According to one of its objects, the present invention relates to an engineered glomerular endothelial cell comprising a reduction in expression of a human leukocyte antigen (HLA).

As shown in the Examples, the inventors have unexpectedly observed that it was possible to obtain genetically engineered human glomerular endothelial cells, in particular genetically engineered conditionally immortalized human glomerular endothelial cells (CiGEnC) not expressing anymore the HLA class I and/or class II molecules.

As shown in the Examples, using the CRISPR-Cas9 technology it has been possible to delete the B2M and CIITA genes in the CiGEnC cells to suppress the expression of class I and II HLA antigens without otherwise affecting the properties, such as growth or morphology of the cells.

Further, as shown in the Examples, the inventors have unexpectedly observed that the obtained genetically engineered CiGEnCΔHLA cells have revealed useful to be implemented in diagnostic methods, such as in vitro methods for determining the likelihood of occurrence of a non-HLA antibody mediated rejection against a renal allograft.

Further, as shown in the Examples, the inventors have unexpectedly observed that it was possible to use, as a biomarker, a ratio of a first quantification of a predetermined signal obtained from antibodies bound to an engineered glomerular endothelial cell as disclosed herein, said antibodies being from a blood sample of an individual presumed to contain non-HLA antibodies, over a second quantification of a predetermined signal obtained from engineered human glomerular endothelial cells as disclosed herein, or obtained according to the method as disclosed herein, in absence of non-HLA antibodies. The biomarker may be for use in a method for determining the likelihood of occurrence of a non-HLA antibody mediated rejection against a renal allograft in an individual in need thereof.

In one embodiment, the HLA may be a HLA of class I, a HLA of class II, or a combination thereof. In one example, the genetically engineered glomerular endothelial cell may comprise a reduction, such as a suppression, in expression of human leukocyte antigen of class I and of class II.

The suppression in the expression of a HLA may be a suppression of the expression at the cell surface.

In some embodiments, the engineered glomerular endothelial cell may comprise a reduction in expression of the beta-2 microglobuline (B2M) protein and/or the class II transactivator (CIITA) protein.

The cell may comprise a reduction in expression of a polynucleotide encoding the beta-2 microglobuline (B2M) protein. The cell may comprise a reduction in expression of a polynucleotide encoding the class II transactivator (CIITA) protein.

The reduction in the expression may be mediated by gene editing or RNA interference (RNAi)-mediated gene silencing.

The reduction in the expression may be mediated by CRISPR/Cas9, adenovirus, lentivirus, and/or adeno-associated virus and/or a combination thereof.

The reduction in the expression may be mediated by CRISPR/Cas9 gene editing.

The reduction in the expression may be mediated by adenovirus, lentivirus, and/or adeno-associated virus mediated RNA interference, and/or a combination thereof.

In some embodiments, the engineered glomerular endothelial cell may comprise a disruption in a gene encoding B2M and/or in a gene encoding CIITA.

In some embodiments, the reduction in the expression may be mediated by a CRISPR/Cas9 gene disruption of a gene encoding B2M and/or a gene encoding CIITA.

The disruption may be in exon 1 and/or in exon 2 of the gene encoding B2M.

Alternatively, or in combination, the disruption may be in exon 2 and/or in exon 3 of a gene encoding CIITA.

In some embodiments, the engineered glomerular endothelial cell may be a CiGEnCΔHLA. In one embodiment, a CiGEnCΔHLA cell line was deposited on Jul. 2, 2021, at the Institut Pasteur under the Budapest Treaty for the International Recognition of the Deposit of Microorganisms for the Purposes of Patent Procedure, with the reference CNCM I-5707.

According to another of its objects, the invention relates to a method for producing an engineered glomerular endothelial cell as disclosed herein, comprising at least a step of reducing expression of a human leukocyte antigen (HLA).

In one embodiment of the method for producing an engineered glomerular endothelial cell as disclosed herein, the reduction in the expression of a human leukocyte antigen (HLA) may be mediated by CRISPR/Cas9 gene editing, adenovirus, lentivirus, and/or adeno-associated virus mediated RNA interference, and/or a combination thereof.

In the method for producing an engineered glomerular endothelial cell as disclosed herein, the reduction in the expression of a human leukocyte antigen (HLA) may be mediated by a CRISPR/Cas9 gene disruption of a gene encoding B2M and/or a gene encoding CIITA.

In the method for producing an engineered glomerular endothelial cell as disclosed herein, the cell may comprise a disruption in a gene encoding B2M and/or in a gene encoding CIITA.

In the method for producing an engineered glomerular endothelial cell as disclosed herein, the disruption may be in exon 1 and/or in exon 2 of the gene encoding B2M.

In the method for producing an engineered glomerular endothelial cell as disclosed herein, the disruption may be in exon 2 and/or in exon 3 of a gene encoding CIITA.

According to one of its objects, the present invention relates to an in vitro diagnostic method. The method may be an in vitro method for determining the likelihood of occurrence of a non-HLA antibody mediated rejection against a renal allograft in an individual in need thereof, comprising at least the steps of:
a) incubating at least one engineered human glomerular endothelial cell as disclosed herein, or obtained as disclosed herein, with a blood sample of said individual, said blood sample being presumed to contain non-HLA antibodies,
b) obtaining a quantification of a predetermined signal of non-HLA antibodies bound to said engineered human glomerular endothelial cell,
c) comparing the quantification obtained at step b) with a predetermined reference value, and
d) wherein a quantification obtained at step b) greater than the predetermined reference value indicates that said individual is at risk of a non-HLA antibody mediated rejection against a renal allograft.

Since they are compared one to another, the quantification obtained at step b) and the predetermined reference value are necessary of same nature, e.g., an absolute fluorescence intensity, a geometric mean of fluorescence, or a ratio of a measured fluorescence over a control fluorescence.

In some embodiments, the method may be an in vitro method for determining the likelihood of occurrence of a non-HLA antibody mediated rejection against a renal allograft in an individual in need thereof, comprising at least the steps of:
a) incubating at least one engineered human glomerular endothelial cell as disclosed herein, or obtained as disclosed herein, with a blood sample of said individual, said blood sample being presumed to contain non-HLA antibodies,
b) obtaining a quantification of non-HLA antibodies bound to said engineered human glomerular endothelial cell,
c) comparing the quantification obtained at step b) with a predetermined reference value, and
d) wherein a quantification of non-HLA antibodies bound to said engineered human glomerular endothelial cell greater than the predetermined reference value indicates that said individual is at risk of a non-HLA antibody mediated rejection against a renal allograft.

The individual's blood sample may be selected in the group consisting of whole blood, blood plasma and blood serum. For example, the blood sample may be selected in the group consisting of blood plasma and blood serum.

The individual may be selected from the group consisting of (i) a candidate individual for a renal allograft and (ii) a recipient of a renal allograft.

In some embodiments, the quantification at step b) may be obtained with a labeled anti-human immunoglobulin antibody, or a fragment thereof.

The label of said labelled anti-human immunoglobulin antibody, or fragment thereof, may be selected in the group consisting of a fluorescent molecule, a radioisotope, an enzyme, a biotin, or a streptavidin.

In some embodiments, the predetermined reference value of step c) may be obtained by quantification of a predetermined signal measured from engineered human glomerular endothelial cells as disclosed herein, or obtained according to the method as disclosed herein, in absence of non-HLA antibodies. To obtain such signal, the cells may be incubated in a medium not containing any non-HLA antibodies, such as a serum of a healthy volunteer, or alternatively not containing any antibodies, such as a buffer.

The predetermined signal which is measured is preferably of the same nature than the predetermined signal measured at step b) to quantify non-HLA antibodies from a blood sample of an individual to be tested which are bound to the cells.

For example, a predetermined reference value of step c) may be obtained by quantifying a signal obtained from engineered human glomerular endothelial cells present in a buffer not containing any non-HLA antibodies and having been contacted with a labeled anti-human immunoglobulin antibody.

In some other embodiments, the predetermined reference value of step c) may be obtained by incubating at least an engineered human glomerular endothelial cell as disclosed herein, or obtained according to a method as disclosed herein, with at least a blood sample of an individual known to not contain non-HLA antibodies and quantification of antibodies bound to said engineered human glomerular endothelial cell.

In some embodiments, the predetermined reference value of step c) may be obtained by quantification of antibodies bound to at least an engineered human glomerular endothelial cell as disclosed herein or obtained according to the method as disclosed herein, said antibodies being from a blood sample of an individual known to not contain non-HLA antibodies.

A blood sample of an individual known to not contain non-HLA antibodies is a control or reference blood sample. Such blood sample is presumed to contain antibodies other than non-HLA antibodies.

In some embodiments, the predetermined reference value of step c) may be a ratio of quantifications, said ratio being equal to a quantification of a predetermined signal of antibodies bound to an engineered glomerular endothelial cell as disclosed herein, said antibodies being from a blood sample of an individual presumed to contain non-HLA antibodies, over a quantification of a predetermined signal measured from engineered human glomerular endothelial cells as disclosed herein or obtained according to the method as disclosed herein, in absence of non-HLA antibodies.

In some embodiments, the predetermined reference value of step c) may be a ratio of quantifications of antibodies, said ratio being equal to a quantification of antibodies bound to an engineered glomerular endothelial cell as disclosed herein, said antibodies being from a blood sample of an individual presumed to contain non-HLA antibodies, over a quantification of antibodies bound to an engineered glomerular endothelial cell as disclosed herein, said antibodies being from a blood sample of an individual known to not contain non-HLA antibodies.

The quantification of antibodies bound to an engineered glomerular endothelial cell according as disclosed herein may be a geometric mean of fluorescence intensity.

A ratio of quantifications of antibodies bound to an engineered glomerular endothelial cell as disclosed herein may be a ratio of geometric means of fluorescence intensity.

A ratio of geometric means of fluorescence intensity may be within a range from about 1.20 to about 3.50, or from about 1.20 to about 3.20, or from about 1.20 to about 3.00, or from 1.20 to about 2.80, or from about 1.30 to about 2.20, or from about 1.40 to about 2.10, from about 1.50 to about 2.00, or from about 1.50 to about 1.90, or from about 1.60 to about 1.80. For example, the ratio may be about 1.87 or about 2.50.

In some embodiments, the cells used in the diagnostic methods disclosed herein may be in suspension.

In some embodiments, the cells used in the diagnostic methods disclosed herein may be adhered to a support.

The cells may be suspension or adhered to a support bathed in a physiologically acceptable buffer. A physiologically acceptable buffer may be a phosphate buffer. A physiologically acceptable buffer may comprise at least one selected in the group consisting of a chelating agent, an isotonic agent, a blocking protein.

According to another of its objects, the invention relates to a method for determining the likelihood of occurrence of a non-HLA antibody mediated rejection against a renal allograft in an individual in need thereof and administering a treatment against a non-HLA antibody mediated rejection against a renal allograft in said individual in need thereof, the method comprising at least the steps of:
i) carrying out the in vitro diagnostic method as disclosed herein, and
ii) administering to said individual a treatment for preventing and/or reducing a non-HLA antibody mediated rejection against a renal allograft if said patient is determined to be at risk of a non-HLA antibody mediated rejection against a renal allograft.

A suitable treatment may be selected among immunosuppressant drugs, plasma exchanges; immuno-adsorptions; intravenous immune globulins; or drugs targeting antibodies, B lymphocytes or plasma cells depleting agents.

According to another of its objects, the invention relates to an engineered human glomerular endothelial cell comprising a reduction in expression of a human leukocyte antigen (HLA) for use in a method for determining the likelihood of occurrence of a non-HLA antibody mediated rejection against a renal allograft in an individual in need thereof.

According to another of its objects, the invention relates to a ratio of quantifications, said ratio being equal to a ratio of a first quantification of a predetermined signal obtained from antibodies bound to an engineered glomerular endothelial cell as disclosed herein, said antibodies being from a blood sample of an individual presumed to contain non-HLA antibodies, over a second quantification of a predetermined signal obtained from engineered human glomerular endothelial cells as disclosed herein, or obtained according to the method as disclosed herein, in absence of non-HLA antibodies, as a biomarker for use in a method for determining the likelihood of occurrence of a non-HLA antibody mediated rejection against a renal allograft in an individual in need thereof.

According to another of its objects, the invention relates to a kit for determining the likelihood of occurrence of a non-HLA antibody mediated rejection against a renal allograft in an individual in need thereof, the kit comprising:
(i) at least one engineered human glomerular endothelial cell as disclosed herein, and
(ii) at least one mean to detect and quantify antibodies bound on said human glomerular endothelial cell.

The bound antibodies are non-HLA antibodies.

The kit as disclosed herein may further comprise at least one instruction to implement an in vitro diagnostic method as disclosed.

The mean to detect and quantify antibodies bound on said human glomerular endothelial cell may be a labeled anti-human immunoglobulin antibody, or a fragment thereof.

The kit as disclosed herein may further comprise an instruction to compare a quantification of a predetermined signal of non-HLA antibodies bound to said engineered human glomerular endothelial cell, said non-HLA antibodies being obtained from an isolated biological sample from an individual, with a predetermined reference value, wherein the predetermined reference value is a ratio of a first quantification of a predetermined signal obtained from antibodies bound to an engineered glomerular endothelial cell comprising a reduction in expression of a human leukocyte antigen (HLA), said antibodies being from a blood sample of an individual known to contain non-HLA antibodies over a second quantification of a predetermined signal obtained from an engineered glomerular endothelial cell comprising a reduction in expression of a human leukocyte antigen (HLA), in absence of non-HLA antibodies.

According to another of its objects, the invention relates to a use of a kit as disclosed herein for determining the likelihood of occurrence of a non-HLA antibody mediated rejection against a renal allograft in an individual in need thereof.

### DESCRIPTION OF THE FIGURES

FIG. 1 represents the B2M and CIITA ablation by CRISPR/Cas9 to generate CiGEnCΔHLA cells. In FIGS. 1a to 1d unmodified CiGEnC cells were transfected with 2 different gRNAs targeting exon 1 and exon 2 B2M loci and an active Cas9 protein. After 5 days, the cells were stimulated with IFN-γ before FACS sorting. FIG. 1a: Schematic view. FIG. 1b: FACS histograms showing HLA-I expression before (left) and after (middle) transfection and after cell sorting (right). FIG. 1c: TIDE analysis of CiGEnCΔB2M cells clonally sorted by single-cell FACS, expanded and sequenced across gRNA target sites. The percentage of sequences with indels is represented. FIG. 1d: Indel characterization across B2M exon 1 and exon 2. The more frequent sequences found are described. FIGS. 1e to 1g: CiGEnCΔB2M cells were transfected with 2 different gRNAs targeting exon 2 and exon 3 CIITA loci and an active Cas9 protein. After 5 days, the cells were stimulated with IFN-γ before FACS sorting. FIG. 1e: Schematic view. FIG. 1f: TIDE analysis of CiGEnCΔHLA cells clonally sorted by single-cell FACS, expanded and sequenced across gRNA target sites. The percentage of sequences with indels is represented. FIG. 1g: Indel characterization across CIITA exon 2 and exon 3. The more frequent sequences found are described.

FIG. 2 represents the loss of HLA antigen expression in CiGEnCΔHLA cells. FIG. 2a: RT-qPCR analysis of B2M, CIITA, HLA-DR, and CXCL10 in unmodified CiGEnC and CiGEnCΔHLA cells with and without 24 h of cytokine stimulation. The results are shown as the relative expression of the genes normalized to GAPDH expression. FIG. 2b: Representative dot plots of FACS analysis of HLA-ABC and HLA-DR expression in unmodified CiGEnC (top panels) and CiGEnCΔHLA (lower panels) cells with (right panels) and without (left panels) 48 h of cytokine stimulation. FIG. 2c: FACS analysis of HLA-ABC (top panel) and HLA-DR (lower panel) expression in unmodified CiGEnC and CIGEnCΔHLA cells with and without 48 h of cytokine stimulation. Expression is presented as the relative fluorescence intensity (RFI) calculated by subtracting the mean fluorescence intensity (MFI) of the corresponding isotype control. FIG. 2d: Immunofluorescence analysis of HLA-ABC (yellow staining) and HLA-DR, HLA-DP and HLA-DQ (red staining) expression in unmodified CiGEnC and CiGEnCΔHLA cells with and without 48 h of cytokine stimulation. DAPI (blue staining) was used as a nuclear counterstain. FIG. 2e: Representative dot plots of FACS analysis of HLA-ABC and HLA-A2 expression in unmodified CiGEnC and CiGEnCΔHLA cells. FIGS. 2f to 2g: Unmodified CiGEnC or CiGEnCΔHLA cells were cocultured with cytotoxic anti-HLA-A2 CAR T cells. FIG. 2g: The normalized cell index (mean±standard error) from three independent experiments is shown.

FIG. 3 represents that CRISP/Cas9 editing does not impair the endothelial phenotype of CiGEnCΔHLA cells. FIG. 3a: RT-qPCR analysis of vWF, KDR, CDH5 and TEK after cell differentiation of unmodified CiGEnC, CiGEnCΔHLA and HK2 cells. The results are shown as the relative expression of the genes normalized to GAPDH expression (n=3 independent experiments). FIG. 3b: Representative dot plots of FACS analysis of VE cadherin and ICAM2 expression (upper panels) and Tie2 and VEGFR2 expression (lower panels) in HRECs (left panels), unmodified CiGEnC cells (middle panels) and CiGEnCΔHLA cells (right panels). FIG. 3c: FACS analysis of VEGFR2, Tie2, VE cadherin and ICAM2 in HRECs and unmodified CiGEnC and CiGEnCΔHLA cells. Expression is presented as the relative fluorescence intensity (RFI) calculated by subtracting the mean fluorescence intensity (MFI) of the corresponding isotype control. FIG. 3d: Immunofluorescence analysis of PECAM1 (green staining), VE cadherin (purple staining) and ICAM2 (white staining) expression in unmodified CiGEnC (top panels) and CiGEnCΔHLA (lower panels) cells. DAPI (blue staining) was used as a nuclear counterstain. FIG. 3e: CiGEnCΔHLA cells maintained morphological features of unmodified CiGEnC cells both at the permissive temperature (33ºC, upper panels) and at a nonpermissive temperature (37° C., lower panels). FIG. 3f: Cell proliferation at 33° C. was analyzed for 55 h using an IncuCyte system. The mean±standard error of 3 independent experiments is shown.

FIG. 4 represents the presence of non-HLA Abs before transplantation is associated with retransplantation status. FIG. 4a: Design of the observational cohort study. FIG. 4b: Schematic view of the non-HLA antibody detection ion assay (NHADIA) process (left) and histograms showing the NHADIA results of a negative control and a patient with serum containing non-HLA Abs. FIG. 4c: Univariate (top panel) and multivariate (lower panel) linear regression analyses of pretransplant determinants of NHADIA results measured at the time of transplantation. FIG. 4d: Distribution of the normalized NHADIA results obtained with pretransplant serum samples from patients awaiting a first transplantation (top panel) or re-transplantation (lower panel).

FIG. 5 represents that the pretransplant NHADIA result is associated with ABMRh lesions at 3 months post-transplantation. FIG. 5a: Dendrogram representations of unsupervised hierarchical clustering analysis of NHADIA quartiles and Banff elementary lesions observed at 3 months after transplantation. The vertical axis of the dendrogram represents the distance or dissimilarity between clusters. FIG. 5b: Percentages of 3-month allograft biopsies with glomerulitis, peritubular capillaritis, C4d staining, microvascular inflammation or ABMRh lesions according to NHADIA tertiles (left panels, P values by chi-2 tests) and the mean±SEM values of the NHADIA results according to the corresponding histological features (right panels, P values by the Kruskal-Wallis test). FIG. 5c: Multivariate logistic regression analysis of pretransplant immunological determinants of ABMRh.

FIG. 6 represents that the pretransplant NHADIA result predicts ABMRh. FIG. 6a: Kaplan-Meier representation of the cumulative incidence of ABMRh according to pretransplant NHADIA results. Data are based on 389 kidney transplant recipients. FIG. 6b: Multivariate Cox analysis of the risk of ABMRh according to the pretransplant HLA-DSAs status and NHADIA status. FIG. 6c: Kaplan-Meier representation of the cumulative incidence of ABMRh according to the pretransplant NHADIA status and HLA-DSA status. FIGS. 6b to 6c: Data are based on 386 kidney transplant recipients due to missing HLA-DSAs data. ABMR: antibody-mediated rejection, ABMRh: ABMR histological lesions, HLA-DSAs: anti-HLA donor-specific antibodies, MVI: microvascular inflammation, NHADIA: non-HLA detection assay, TCMR; T cell-mediated rejection. FIG. 6d: Log-rank P values of the comparison of the cumulative incidence of ABMRh according to various NHADIA thresholds.

FIG. 7 represents changes in the diagnostic categories of kidney allograft biopsies according to the Banff′13 and Banff′17 classifications for ABMR and suggestions for classification improvement. FIG. 7a: NHADIA status in kidney transplant recipients with or without TCMR or ABMR according to the Banff 2017 classification, ABMRh and MVI (N corresponds to the numbers of patients diagnosed with the corresponding histological features, P values by chi-2 tests). FIG. 7b: All post-transplant biopsies were classified according to Banff 2013 and Banff 2017 classifications. Of these, 754 biopsies were not considered sABMR or ABMR in any classification. The evolution of the diagnostic categorization of the other 179 biopsies across different Banff versions is depicted. The proposed reclassification according to NHADIA status is also depicted. Line colors represent the diagnostic class in the preceding Banff version. ABMR: antibody-mediated rejection, sABMR: suspicious for ABMR, HLA-DSAs: anti-HLA donor-specific antibodies, MVI: microvascular inflammation, NHADIA: non-HLA detection assay.

### DESCRIPTION OF THE SEQUENCES

SEQ ID NO: 1 GAGTAGCGCGAGCACAGCTA represents the B2M crispr RNA (crRNA)-targeting sequence of B2M exon 1.

SEQ ID NO: 2 AGTCACATGGTTCACACGGC represents the B2M crispr RNA (crRNA)-targeting sequence of B2M exon 2.

SEQ ID NO: 3 CATCGCTGTTAAGAAGCTCC represents the CIITA crRNAs targeting sequence of CIITA exon 2.

SEQ ID NO: 4 GATATTGGCATAAGCCTCCC represents the CIITA crRNAs targeting sequence of CIITA exon 3.

SEQ ID NO: 5 ATATAAGTGGAGGCGTCGCG represents a primer B2M Exon 1 Forward.

SEQ ID NO: 6 TGGAGAGACTCACGCTGGAT represents a primer B2M Exon 1 Reverse.

SEQ ID NO: 7 TGTCTTTCAGCAAGGACTGGT represents a primer B2M Exon 2 Forward.

SEQ ID NO: 8 ACCCCACTTAACTATCTTGGGC represents a primer B2M Exon 2 Reverse.

SEQ ID NO: 9 CTGCCTCTTTCCAACACCCT represents a primer CIITA Exon 2 Forward.

SEQ ID NO: 10 CTTCTCCAGCCAGGTCCATC represents a primer CIITA Exon 2 Reverse.

SEQ ID NO: 11 TTTCAGCAGGCTGTTGTGTG represents a primer CIITA Exon 3 Forward.

SEQ ID NO: 12 GCAGCAAAGAACTCTTGCCC represents a primer CIITA Exon 3 Reverse.

SEQ ID NO: 13 TGAGAGTACCAGGTGTGACG represents an off-target site of B2M exon 2 HDHD1P2.

SEQ ID NO: 14 TCGTCGGCAGCGTCGTGCAGTCTGGGATTTGGGA represents a primer HDHD1P2_F.

SEQ ID NO: 15 GAGGGCCGTCTCGTGGGCTCGGTATGAGTGAGAGG represents a primer HDHD1P2_R.

SEQ ID NO: 16 CATCACTGCTAGGAAGCTTCAGG represents an off-target coding site of CIITA exon 2 JMJD4.

SEQ ID NO: 17 TCGTCGGCAGCGTCATCAAAGGCTGCCTGTTCGA represents a primer JMJD4_F.

SEQ ID NO: 18 GTCTCGTGGGCTCGGTGCTCGGGCATCAACTTTGA represents a primer JMJD4_R.

SEQ ID NO: 19 GATATCTGCATAACCCTTCCAGG represents an off-target coding site of CIITA exon 3 OSTF1.

SEQ ID NO: 20 TCGTCGGCAGCGTCGGGAGATACAGCTTTGCATGC represents a primer OSTF1_F.

SEQ ID NO: 21 AAGACCAGTCTCGTGGGCTCGGTTCAGGGCAAGCA represents a primer OSTF1_R.

SEQ ID NO: 22 5′-CCACATCGCTCAGACACCAT-3′ represents a primer sense.

SEQ ID NO: 23 5′-TGACCAGGCGCCCAATA-3′ represents a primer antisense.

SEQ ID NO: 24 5′-FAM-AGTCAACGGATTTGGTC-MGB-3′ represents a probe.

SEQ ID NO: 25 5′-TGTCCACGTGTTGAGATCATTG-3′ represents a primer sense.

SEQ ID NO: 26 5′-GGCCTTCGATTCTGGATTCA-3′ represents a primer antisense.

SEQ ID NO: 27 5′-FAM-TACAATGAAAAAGAAGGGTGAGAA-MGB-3′ represents a probe.

SEQ ID NO: 28 B2M Exon 1 gRNA PAMATCGACGAGCGCGATGAG

SEQ ID NO: 29 28 B2M Exon 1 5′ TCCGTGGCCTTAGCTGTGCTCGCGCTACTC 3′

SEQ ID NO: 30 28 B2M Exon 1 5′ TCCGTGGCCTGCTGTGCTCGCGCTACTC 3′

SEQ ID NO: 31 28 B2M Exon 1 5′ TCCGTGGCCTAGCTGTGCTCGCGCTACTC 3′

SEQ ID NO: 32 B2M Exon 2 gRNA PAMCACACTTGGTACACTGACT

SEQ ID NO: 33 B2M Exon 2 5′ TGAGTATGCCTGCCGTGTGAACCATGTGACTGA 3′

SEQ ID NO: 34 B2M Exon 2 5′ TGAGTGTGAACCATGTGACTGA 3′

SEQ ID NO: 35 B2M Exon 2 5′ TGAGTATGCCTGCCGTGACTGA 3′

SEQ ID NO: 36 B2M Exon 2 5′ TGAGTATGCCTGAACCATGTGACTGA 3′

SEQ ID NO: 37 CIITA EXON 2 gRNA PAMCCTCGAAGAATTGTCGCTAC

SEQ ID NO: 38 CIITA EXON 2 5′ GCTACCTGGAGCTTCTTAACAGCGATG 3′

SEQ ID NO: 39 CIITA EXON 2 5′ GCTTCTTAACAGCGATG 3′

SEQ ID NO: 40 CIITA EXON 2 5′ GCTACCTGGACTTCTTAACAGCGATG 3′

SEQ ID NO: 41 CIITA EXON 3 gRNA PAMCCCTCCGAATACGGTTATAG

SEQ ID NO: 42 CIITA EXON 3 5′ GAGACCAGGGAGGCTTATGCCAATATC 3′

SEQ ID NO: 43 CIITA EXON 3 5′ GAGACCAGGGCTTATGCCAATATC 3′

SEQ ID NO: 44 CIITA EXON 3 5′ GAGACCAGGGGCTTATGCCAATATC 3′

SEQ ID NO: 45 CIITA EXON 3 5′ GAGACCAGGGAAGGCTTATGCCAATATC 3′

### DETAILED DESCRIPTION

### Definitions

It must be noted that as used herein and in the appended claims, the singular forms “a”, “an”, and “the” include plural referents unless the context clearly dictates otherwise. Thus, for example, reference to “an antibody” includes a plurality of such antibodies, and so forth.

The terms “about” or “approximately” as used herein refer to the usual error range for the respective value readily known to the skilled person in this technical field. Reference to “about” a value or parameter herein includes (and describes) embodiments that are directed to that value or parameter per se. In some embodiments, the term “about” refers to +10% of a given value. However, whenever the value in question refers to an indivisible object, such as a molecule or other object that would lose its identity once subdivided, then “about” refers to +1 of the indivisible object.

It is understood that aspects and embodiments of the present disclosure described herein include “having,” “comprising,” “consisting of,” and “consisting essentially of” aspects and embodiments. The words “have” and “comprise,” or variations such as “has,” “having,” “comprises,” or “comprising,” will be understood to imply the inclusion of the stated element(s) (such as a composition of matter or a method step) but not the exclusion of any other elements. The term “consisting of” implies the inclusion of the stated element(s), to the exclusion of any additional elements. The term “consisting essentially of” implies the inclusion of the stated elements, and possibly other element(s) where the other element(s) do not materially affect the basic and novel characteristic(s) of the disclosure. It is understood that the different embodiments of the disclosure using the term “comprising” or equivalent cover the embodiments where this term is replaced with “consisting of” or “consisting essentially of”.

Within the invention, “HLA of class I” and “HLA of class II” intend to refer to a group of related proteins that are encoded by the major histocompatibility complex (MHC) gene complex in humans. HLAs corresponding to MHC class I (A, B, and C), all of which are the HLA Class1 group, present peptides from inside the cell. HLAs corresponding to MHC class II (DP, DM, DO, DQ, and DR) present antigens from outside of the cell to T-lymphocytes. Any cell displaying some other HLA type is “non-self” and is seen as an invader by the body's immune system, resulting in the rejection of the tissue bearing those cells. This is particularly important in the case of transplanted tissue, because it could lead to transplant rejection.

Within the meaning of the invention, the expressions “non-HLA antibody” or “non-anti-HLA antibody” are used interchangeably and intend to refer to immunoglobulins able to bind cell surface antigens which are not a human leukocyte antigen (HLA) and which do not bind to HLA. As examples of cell surface antigens which may be bound by the non-HLA antibodies, one may cite the antigens selected in the group consisting of ZG16B, LMOD1, BMPR1A, MBP, APEX2, COR02A, CCBE1, EPHA5, TLE4, EV15L, PLEKHA1, TGM2, ERC1, ZBTB14, TMOD2, MAPK1IP1L, TFEB, PFKFB2, EPHB6 and PNMA2.

HLA-antibodies, to the opposite of non-HLA antibodies, are antibodies specifically binding HLA.

Within the meaning of the invention, the terms “patient”, “subject” “recipient” or “individual” are used interchangeably and intends to refer to a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In some exemplary embodiments, the individual or recipient is a human. Those terms intend to refer to individual in need of receiving or having received a kidney transplantation.

As used herein, the terms “prevent”, “preventing” or “delay progression of” (and grammatical variants thereof) with respect to a disease or disorder relate to prophylactic treatment of the disease or the disorder, e.g., in an individual suspected to have the disease or the disorder, or at risk for developing the disease or the disorder. Prevention may include, but is not limited to, preventing or delaying onset or progression of the disease and/or maintaining one or more symptoms of the disease or disorder at a desired or sub-pathological level. The term “prevent” does not require the 100% elimination of the possibility or likelihood of occurrence of the event. Rather, it denotes that the likelihood of the occurrence of the event has been reduced in the presence of a composition or method as described herein.

As used herein, in the context of an immune response elicitation, the terms “treat”, “treatment”, “therapy” and the like refer to the administration or consumption of a composition as disclosed herein with the purpose to cure, heal, alleviate, relieve, alter, remedy, ameliorate, improve, or affect a disease or a disorder, the symptoms of the condition, or to prevent or delay the onset of the symptoms, complications, or otherwise arrest or inhibit further development of the disorder in a statistically significant manner. Also, as used herein, in the context of the present disclosure, the terms “treat”, “treatment” and the like refer to relief from or alleviation of pathological processes mediated by non-HLAs antibodies mediated kidney transplant rejection. In the context of the present disclosure, insofar as it relates to any of the other conditions recited herein, the terms “treat”, “treatment”, and the like refer to relieving or alleviating one or more symptoms associated with such condition.

Within the meaning of the invention, the expression “reduction in the expression of” in connection with an item intends to mean that the expression of the concerned reduced item is below the normal or reference expression of the concerned item. The reduction of expression may be partial or total. A reduction of the expression of a concerned item refers to an expression that is reduced, diminished, or suppressed in a manner such that the concerned item cannot exerts its functions and/or cannot be detected in the cell compartment where it is usually present using conventional methods in the field.

In the invention, the expression of a HLA at a cell surface may be reduced such that it cannot be detected at the surface of a cell using conventional means and methods in the field, for example as disclosed in the Examples. An expression which is not detectable anymore, according to conventional means and methods in the field, is considered as being suppressed.

The reduction or suppression of expression of an item may be direct or indirect. The reduction or suppression of expression of an item may be exerted at the polynucleotide or the protein level.

In the invention, the reduction or suppression of expression of a HLA at the cell surface may be indirect, that is subsequence to the reduction or suppression of expression of B2M and/or CTIIA.

In the invention, the reduction or suppression of expression of B2M and/or CTIIA may be direct, for example subsequent to ablation or disruption of the corresponding gene or blocking of the gene expression or mRNA translation, or even by accelerating degradation of the corresponding protein. For example, the reduction or suppression of expression of B2M and/or CTIIA may be subsequent to ablation or disruption of the corresponding gene.

Within the disclosure, the term “significantly” used with respect to change intends to mean that the observe change is noticeable and/or it has a statistic meaning.

Within the disclosure, the term “substantially” used in conjunction with a feature of the disclosure intends to define a set of embodiments related to this feature which are largely but not wholly similar to this feature. The difference between the set of embodiments related to the given feature and the given feature is such that in the set of embodiments, the nature and function of the given feature is not materially affected.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention. All publications mentioned herein are incorporated herein by reference to disclose and describe the methods and/or materials in connection with which the publications are cited.

The list of sources, ingredients, and components as described hereinafter are listed such that combinations and mixtures thereof are also contemplated and within the scope herein.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All lists of items, such as, for example, lists of ingredients, are intended to and should be interpreted as Markush groups. Thus, all lists can be read and interpreted as items “selected from the group consisting of” the list of items “and combinations and mixtures thereof.”

Referenced herein may be trade names for components including various ingredients utilized in the present disclosure. The inventors herein do not intend to be limited by materials under any particular trade name. Equivalent materials (e.g., those obtained from a different source under a different name or reference number) to those referenced by trade name may be substituted and utilized in the descriptions herein.

### Engineered Glomerular Endothelial Cells

Glomerular endothelial cells refer to highly flattened, non-proliferative cells that provide an anticoagulant surface, participate in forming a barrier to filtration, and produce vasoactive and growth regulating mediator. During glomerular development, and in response to some forms of immune-mediated injury, glomerular endothelial cells lose their flattened appearance and become activated. The glomerular endothelial cells are the endothelial cells lining the capillaries of the glomerulus of the kidney.

The glomerulus is a tuft of capillaries located within Bowman's capsule within the kidney. Blood enters the capillaries of the glomerulus by a single arteriole called an afferent arteriole and leaves by an efferent arteriole. The capillaries consist of a tube lined by endothelial cells with a central lumen. The gaps between these endothelial cells are called fenestrae. The walls have a unique structure: there are pores between the cells that allow water and soluble substances to exit, and after passing through the glomerular basement membrane, and between podocyte foot processes, enter the capsule as ultrafiltrate.

The glomerular endothelial cells are involved in regulating the high flux filtration of fluid and small solutes. During filtration, plasma passes through the fenestrated endothelium and basement membrane before it reaches the slit diaphragm, a specialized type of intercellular junction that connects neighboring podocytes.

Due to their highly specialized role and function in the glomerulus and in the kidney filtration, it has appeared to the inventors that the glomerular endothelial cells presented unique features that could not be exhibited by other type of endothelial cells either from other vessel types or from other organs.

The present invention relates to a genetically engineered glomerular endothelial cell comprising a reduction in expression of a human leukocyte antigen (HLA).

The genetically engineered glomerular endothelial cell as disclosed herein is an isolated cell.

The expressions “genetically engineered” or “genetically modified” are used interchangeably to refer to glomerular endothelial cells that by the hand of man have been changed with respect to the expression and/or activity of at least one gene or other genetic element that is endogenous to the cell.

The glomerular endothelial cell (GEnC) to be genetically engineered may be any of any origin, provided it is a glomerular endothelial cell. In one embodiment, the glomerular endothelial cell may be from a primate, such as a human.

The engineered glomerular endothelial cell is a human engineered glomerular endothelial.

The cell may be a primary or an immortalized cell line. Primary or immortalized cell lines may be obtained according to any known method in the art.

As example of primary GEnC line usable for the invention one may refer to Human Renal Glomerular Endothelial Cells (HRGEC) sold by ScienceCell under Catalog reference #4000 or the Human Kidney Glomerular Endothelial Cells sold by Novabiosis under Catalog reference #3041.

In one embodiment, the cell line may be an immortalized cell line, for example a conditionally immortalized glomerular endothelial cell line (CiGEnC). This cell line has key characteristics of GEnCs, including expression of markers such as PECAM1, ICAM2, VEGFR2, vWF, and, uniquely for a cell line, upregulates fenestrations in response to VEGF. This cell line was described by Satchell, S. C. et al. (Kidney Int. 69, 1633-1640 (2006).).

The glomerular endothelial cells of the invention are genetically engineered to obtain a reduction in expression of a human leukocyte antigen (HLA).

In some embodiments, the glomerular endothelial cell (GEnC) may be engineered to reduce the expression, for example to suppress the expression, of the HLA. The HLA may be HLA of class I, HLA of class II, or a combination thereof. In one example, the genetically engineered glomerular endothelial cell may comprise a reduction, such as a suppression, in expression of human leukocyte antigen of class I and of class II. The suppression in the expression of a HLA may be a suppression of the expression at the cell surface.

As example of HLA of class I or of class II one may cite the human leukocyte antigen (HLA)-A, HLA-B, HLA-C, HLA-DP, HLA-DQ, HLA-DR.

In some embodiments, the engineered glomerular endothelial cell may comprise a reduction in expression of the beta-2 microglobuline (B2M) protein and/or the class II transactivator (CIITA) protein.

The cell may comprise a reduction in expression of a polynucleotide encoding the beta-2 microglobuline (B2M) protein. The cell may comprise a reduction in expression of a polynucleotide encoding the class II transactivator (CIITA) protein.

A reduction in expression of the beta-2 microglobuline (B2M) protein and/or the class II transactivator (CIITA) protein may be obtained with any method known in the art.

In one embodiment, the glomerular endothelial cells of the invention are genetically engineered to obtain a suppression of expression of HLA I and HLA II.

The suppression of expression of HLA I and HLA II may be subsequent to the disruption of the genes encoding for B2M and CTIIA.

In one embodiment, the reduction in the expression may be mediated by gene editing.

The reduction in the expression may be mediated by adenovirus, lentivirus, and/or adeno-associated virus mediated RNA interference, and/or a combination thereof.

In one embodiment, the reduction in the expression may be mediated by a targetable nuclease, such as CRISPR/Cas9, or by RNA mediated interference induced with adenovirus, lentivirus, and/or adeno-associated virus and/or a combination thereof.

For example, suitable methods may include obtaining a targetable nuclease (e.g., as a protein or a gene for a nuclease). Any suitable nuclease can be used such as zinc-finger nucleases (ZFNs), transcription activator-like effector nucleases (TALENs), clustered regularly interspaced short palindromic repeat (CRISPR) nucleases, meganucleases, other endo- or exo-nucleases, or combinations thereof.

In certain embodiments, the disclosure may use a CRISPR associated nuclease.

The formation of a CRISPR complex (which is made of a guide sequence hybridized to a target sequence and complexed with one or more Cas proteins) will cause cleavage of one or both strands in or near the target sequence.

CRISPR may use separate guide RNAs known as the crRNA and tracrRNA.

These two separate RNAs have been combined into a single RNA to enable site-specific mammalian genome cutting through the design of a short guide RNA. A CRISPR-associated nuclease and guide RNA (gRNA) may be synthesized by known methods. For example, Cas9/guide-RNA (gRNA) uses a non-specific DNA cleavage protein Cas9, and an RNA oligo to hybridize to target and recruit the Cas9/gRNA complex.

In one embodiment, a CRISPR-associated (Cas9) nuclease may be used. The reduction in the expression may be mediated by CRISPR/Cas9 gene editing.

Guide RNAs or single guide RNAs may be specifically designed to of the beta-2 microglobuline (B2M) gene and/or the class II transactivator (CIITA) gene. As used herein targeting sequence can mean any combination of gRNA, crRNA, tracrRNA, sgRNA, and others. A CRISPR/Cas9 gene editing complex of the invention works optimally with a guide RNA that targets a gene. Guide RNA (gRNA) (which includes single guide RNA (sgRNA), crisprRNA (crRNA), transactivating RNA (tracrRNA), any other targeting oligo, or any combination thereof) leads the CRISPR/Cas9 complex to a gene of interest in order to cause a gene disruption.

The tracr sequence may comprise or consist of all or a portion of a wild-type tracr sequence and may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. When inducing gene editing in cells, a Cas enzyme, a guide sequence linked to a tracr-mate sequence, and a tracr sequence could be transfected as a reagent complex or provided as polynucleotide sequences operably linked to separate regulatory elements on separate vectors.

A sequence is provided that targets the nuclease to specific targets in the genes encoding B2M and/or CIITA on the GEnC genome.

The sequence may be in the form of DNA that is complementary to guide-RNA, which sequence will be transcribed within the GEnC to provide the final gRNA.

In one embodiment, a DNA vector encoding Cas9, may code for gRNAs that are complementary to specific targets within the genes encoding B2M and/or CIITA.

In some embodiments, the Cas9 nuclease is used to disrupt the genes encoding B2M and/or CIITA, resulting in effective ablation of the genes.

For example, a target for disruption may be in exon 1 and/or in exon 2 of the gene encoding B2M.

For example, a target for disruption may be in exon 2 and/or in exon 3 of a gene encoding CIITA.

For example, the B2M crispr RNA (crRNA)-targeting sequences may include SEQ ID NO: 1 GAGTAGCGCGAGCACAGCTA (B2M exon 1) and SEQ ID NO: 2 AGTCACATGGTTCACACGGC (B2M exon 2).

The CIITA crRNAs targeting sequences may include SEQ ID NO: 3 CATCGCTGTTAAGAAGCTCC (CIITA exon 2) and SEQ ID NO: 4 GATATTGGCATAAGCCTCCC (CIITA exon 3).

The nuclease may be provided in the cells as a protein or as a polynucleotide encoding protein.

The nuclease gene and encoded gRNAs may be provided in a DNA vector, such as a plasmid, a linear DNA, or a viral vector, such as an adenovirus-based vector, and the vector may further optionally include a GEnC-specific inducible promoter. That composition is then introduced into the cells. Any suitable transfection or delivery method may be used. Once in the cell, the genes are expressed and the Cas9 enzyme uses the gRNA to target, and cleave, the genes encoding B2M and/or CIITA.

CRISPR/Cas9/gRNA may be transfected into cells by various methods, including viral vectors and non-viral vectors. Viral vectors may include retroviruses, lentiviruses, adenoviruses, and adeno-associated viruses. It should be appreciated that any viral vector may be incorporated into the present invention to effectuate delivery of the CRISPR/Cas9/gRNA complex into a cell. Some viral vectors may be more effective than others, depending on the CRISPR/Cas9/gRNA complex designed for digestion or incapacitation. In an aspect of the invention, the vectors contain essential components such as origin of replication, which is necessary for the replication and maintenance of the vector in the host cell.

Viral vectors which may be used as delivery vectors to deliver the complexes into a cell may be, for example, a retrovirus, a lentivirus, an adenovirus or a related AAV. In one embodiment, two or more of the elements expressed from the same or different regulatory elements may be combined in a single vector, with one or more additional vectors providing any components of the CRISPR system that are not included in the first vector.

Non-viral vectors delivery of nucleic acids and/or protein which may be used to effectuate a transfection may include lipofection, nucleofection, microinjection, biolistics, virosomes, liposomes, immunoliposomes, polycation or lipid:nucleic acid conjugates, naked DNA, artificial virions, and agent-enhanced uptake of DNA.

Lipofection is described in e.g., U.S. Pat. Nos. 5,049,386, 4,946,787; and 4,897,355, and lipofection reagents are sold commercially (e.g., Transfectam and Lipofectin).

Synthetic vectors are typically based on cationic lipids or polymers which can complex with negatively charged nucleic acids to form particles with a diameter in the order of 100 nm. The complex protects nucleic acid from degradation by nuclease. Moreover, cellular and local delivery strategies have to deal with the need for internalization, release, and distribution in the proper subcellular compartment.

In some embodiments, the Cas9 nuclease is provided as a recombinant protein in combination with the guide RNA with a non-viral vector.

In some embodiments, the reduction in the expression may be mediated by adenovirus, lentivirus, and/or adeno-associated virus mediated RNA interference, and/or a combination thereof.

RNAi molecules can be active for gene silencing, for example, a dsRNA that is active for gene silencing, a siRNA, a micro-RNA, or a shRNA active for gene silencing, as well as a DNA-directed RNA (ddRNA), a Piwi-interacting RNA (piRNA), and a repeat associated siRNA (rasiRNA). Such molecules are capable of mediating RNA interference.

A RNAi molecule of this invention can be targeted to B2M and CTIIA, and any homologous sequences, for example, using complementary sequences or by incorporating non-canonical base pairs, for example, mismatches and/or wobble base pairs, that can provide additional target sequences.

RNAi molecules may be commercially available or may be designed and prepared based on known sequence information, etc. The antisense nucleic acid includes RNA, DNA, PNA, or a complex thereof. As used herein, the DNA/RNA chimera polynucleotide includes a double-strand polynucleotide composed of DNA and RNA that inhibits the expression of a target gene.

In one embodiment, an RNAi molecule may be a siRNA molecule. An siRNA molecule can have a length from about 10-50 or more nucleotides. Commercially available design tools and kits, such as those available from Ambion, Inc. (Austin, TX), and the Whitehead Institute of Biomedical Research at MIT (Cambridge, MA) allow for the design and production of siRNA.

In some embodiments, the genetically engineered glomerular endothelial cell may be a CiGEnC in which the genes encoding for B2M and/or CIITA, and preferably for both B2M and CIITA have been disrupted. Such cell line is designed thereafter: CIGEnCΔHLA. A cell line CiGEnCΔHLA was deposited on Jul. 2, 2021, at the Institut Pasteur under the Budapest Treaty for the International Recognition of the Deposit of Microorganisms for the Purposes of Patent Procedure, with the reference CNCM I-5707.

According to another of its objects, the invention relates to a method for producing an engineered glomerular endothelial cell as disclosed herein, comprising at least a step of reducing expression of a human leukocyte antigen (HLA).

In one embodiment of the method for producing an engineered glomerular endothelial cell as disclosed herein, the reduction in the expression of a human leukocyte antigen (HLA) may be mediated by CRISPR/Cas9, adenovirus, lentivirus, and/or adeno-associated virus and/or a combination thereof.

In one embodiment of the method for producing an engineered glomerular endothelial cell as disclosed herein, the reduction in the expression of a human leukocyte antigen (HLA) may be mediated by CRISPR/Cas9-gene editing, or adenovirus, lentivirus, and/or adeno-associated virus RNA interference (RNAi)-mediated gene silencing, and/or a combination thereof.

In one embodiment, the reduction in the expression of a human leukocyte antigen (HLA) may be mediated by a CRISPR/Cas9 gene disruption of a gene encoding B2M and/or a gene encoding CIITA. For example, the gene disruption may be obtained as generally disclosed above or as disclosed in detail in the Examples.

In the method for producing an engineered glomerular endothelial cell as disclosed herein, the cell may comprise a disruption in a gene encoding B2M and/or in a gene encoding CIITA.

In the method for producing an engineered glomerular endothelial cell as disclosed herein, the disruption may be in exon 1 and/or in exon 2 of the gene encoding B2M.

In the method for producing an engineered glomerular endothelial cell as disclosed herein, the disruption may be in exon 2 and/or in exon 3 of a gene encoding CIITA.

In one embodiment, the method as disclosed herein may comprise a disruption in exon 1 and in exon 2 of the gene encoding B2M, and in exon 2 and in exon 3 of a gene encoding CIITA.

In one object, the invention relates to an engineered glomerular endothelial cell obtained according to the method disclosed herein.

### Diagnostic Methods and Kits for Use Therein

### Methods

According to one of its objects, the present invention relates to an in vitro diagnostic method. The method may be an in vitro method for determining the likelihood of occurrence of a non-HLA antibody mediated rejection against a renal allograft in an individual in need thereof, comprising at least the steps of:
a) incubating at least one engineered human glomerular endothelial cell as disclosed herein, or obtained as disclosed herein, with a blood sample of said individual, said blood sample being presumed to contain non-HLA antibodies,
b) obtaining a quantification of a predetermined signal of non-HLA antibodies bound to said engineered human glomerular endothelial cell,
c) comparing the quantification obtained at step b) with a predetermined reference value, and
d) wherein a quantification obtained at step b) greater than the predetermined reference value indicates that said individual is at risk of a non-HLA antibody mediated rejection against a renal allograft.

In some embodiments, the method may be an in vitro method for determining the likelihood of occurrence of a non-HLA antibody mediated rejection against a renal allograft in an individual in need thereof, comprising at least the steps of:
a) incubating at least one engineered human glomerular endothelial cell as disclosed herein, or obtained as disclosed herein, with a blood sample of said individual, said blood sample being presumed to contain non-HLA antibodies,
b) obtaining a quantification of non-HLA antibodies bound to said engineered human glomerular endothelial cell,
c) comparing the quantification obtained at step b) with a predetermined reference value, and
d) wherein a quantification of non-HLA antibodies bound to said engineered human glomerular endothelial cell greater than the predetermined reference value indicates that said individual is at risk of a non-HLA antibody mediated rejection against a renal allograft.

According to one of its objects, the present invention relates to an in vitro method for determining a likelihood of occurrence of a non-HLA antibody mediated rejection against a renal allograft in an individual in need thereof, the method comprising at least the steps of:
a) incubating at least one engineered human glomerular endothelial cell comprising a reduction in expression of a human leukocyte antigen (HLA) with a blood sample of said individual, said blood sample being presumed to contain non-HLA antibodies,
b) obtaining a quantification of a predetermined signal of non-HLA antibodies bound to said engineered human glomerular endothelial cell,
c) comparing the quantification obtained at step b) with a predetermined reference value, wherein the predetermined reference value is a ratio of a first quantification of a predetermined signal obtained from antibodies bound to an engineered glomerular endothelial cell comprising a reduction in expression of a human leukocyte antigen (HLA), said antibodies being from a blood sample of an individual known to contain non-HLA antibodies over a second quantification of a predetermined signal obtained from an engineered glomerular endothelial cell comprising a reduction in expression of a human leukocyte antigen (HLA), in absence of non-HLA antibodies, and
d) wherein a quantification obtained at step b) greater than the predetermined reference value indicates that said individual is at risk of a non-HLA antibody mediated rejection against a renal allograft.

A diagnostic method as disclosed herein may be used for determining the likelihood of occurrence of antibody-mediated rejection (ABMR).

A diagnostic method as disclosed herein may be used for determining the likelihood of occurrence of ABMR histology (ABMRh).

A diagnostic method as disclosed herein may be used for determining the likelihood of occurrence of an acute microvascular rejection (AMVR) against a renal allograft in a transplanted recipient or in an individual in need to receive a renal allograft transplant.

The in vitro method according to the invention allows to determine the likelihood of occurrence of an acute microvascular rejection (AMVR) against a renal allograft in an individual with an enhanced sensitivity. Preferably, the in vitro method of the invention further allows to reduce the number of false-negative and/or false-positive results in the diagnosis of individuals who are tested to determine whether they are at risk of developing an AMVR.

The individual's blood sample may be selected in the group consisting of whole blood, blood plasma and blood serum. For example, the blood sample may be selected in the group consisting of blood plasma and blood serum.

The individual may be selected from the group consisting of (i) a candidate individual for a renal allograft and (ii) a recipient of a renal allograft. Such individual may be an individual who suffers from a disease which may require a kidney transplant such as diabetes, chronic glomerulonephritis, polycystic kidney disease, sickle cell nephropathy, high blood pressure, severe defects of the urinary tract, or chronic kidney disease.

Step a) of a method disclosed herein comprises incubating the genetically engineered glomerular endothelial cells disclosed herein with a sample of an individual under conditions wherein non-HLA antibodies bind to the cells.

Suitable conditions for antibody-antigen binding depend on several factors such as temperature, pH, ionic strength, concentrations of antigen and antibody, duration of incubations, etc. It is routine work for a skilled person to find suitable conditions for non-HLA antibodies to bind the engineered glomerular cells disclosed herein.

For example, a suitable buffer may be a phosphate buffer, a MES (morpholino-ethanesulfonic acid) buffer, a BIS-TRIS buffer, a citrate buffer, a TRIS-HCl buffer, or a borate buffer. A phosphate buffer may comprise sodium phosphate monobasic and sodium phosphate dibasic.

A buffer may comprise a blocking agent. A blocking agent may be the bovine serum albumin (BSA), non-fat milk, serum (horse or fetal calf), fish gelatin, or casein. A buffer may comprise bovine serum albumin (BSA).

A buffer may comprise a chelating agent. A chelating agent may be trisodium ethylenediamine disuccinate, tetrasodium EDTA, diethylenetriaminepenta acetic acid (DTPA). A chelating agent may be EDTA.

Duration of incubation may range, for example, from 15 min to 1 hour, and may be for example of about 30 min.

A predetermined signal to be quantified at step b) may be any type of suitably detectable signal such as a fluorescent, luminescent, radioactive, or colorimetric signal. Such predetermined signal may be obtained with any suitable label or tag. A suitable label or tag may be attached to the non-HLA antibodies bound to the cells either directly or indirectly, for example by means of a secondary antibody able to bind the Fc part of the non-HLA antibodies.

In some embodiments, the quantification at step b) may be obtained with a labeled anti-human immunoglobulin antibody, or a fragment thereof. In particular, use may be made of secondary antibodies which have been previously labeled and which target anti-human IgGs.

The quantification may be carried with an immunoassay. In some embodiments, an immunoassay may be selected from the group comprising an ELISA, radio-immunoassay, automated immunoassay, cytometric bead assay, and immunoprecipitation assay. In other embodiments, the labeled anti-human immunoglobulin antibody comprises reporter molecule for performing a fluorescently activated cell sorting assay.

The labeled anti-human immunoglobulin antibody may bear a reporter molecule. Numerous labels or reporter molecules may be used, such as:
(a) Radioisotopes, such as ³⁵S, ¹⁴C, ¹²⁵I, ³H, and ¹³¹I. Radioactivity can be measured using scintillation counting. Other radionuclides include ⁹⁹Tc, ⁹⁰Y, ¹¹¹In, ³²P, ¹¹C, ¹⁵O, ¹³N, ¹⁸F, ⁵¹Cr, ⁵⁷To, ²²⁶Ra, ⁶⁰Co, ⁵⁹Fe, ⁵⁷Se, ¹⁵²Eu, ⁶⁷CU, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Mn, ⁵²Tr, and ⁵⁶Fe.
(b) Colloidal gold particles.
(c) Fluorescent or chemiluminescent labels including, but not limited to, rare earth chelates (europium chelates), fluorescein and its derivatives, rhodamine and its derivatives, isothiocyanate, phycoerythrin, phycocyanin, allophycocyanin, o-phthaladehyde, fluorescamine, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, Texas Red, dansyl, Lissamine, umbelliferone, phycocrytherin, phycocyanin, or commercially available fluorophores such SPECTRUM ORANGE® and SPECTRUM GREEN® and/or derivatives of any one or more of the above. The fluorescent labels can be conjugated to the antibody using the techniques disclosed in Current Protocols in Immunology, supra, for example. Fluorescence can be quantified using a fluorimeter.
(d) Enzyme catalyzing a substrate-based colorimetric reaction. Various enzyme-substrate labels are available. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate becomes electronically excited by a chemical reaction and may then emit light which can be measured (using a chemiluminometer, for example) or donates energy to a fluorescent acceptor.

Examples of enzymatic labels include luciferases (e.g., firefly luciferase and bacterial luciferase), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Numerous enzyme-substrate combinations are available to those skilled in the art. Examples of enzyme-substrate combinations are:
(i) Horseradish peroxidase (HRP) with hydrogen peroxidase as a substrate, wherein the hydrogen peroxidase oxidizes a dye precursor, such as, e.g., 3,3′ diamino benzidine (DAB), which produces a brown end product; 3-amino-9-ethylcarbazole (AEC), which upon oxidation forms a rose-red end product; 4-chloro-1-napthol (CN), which precipitates as a blue end product; and p-Phenylenediamine dihydrochloride/pyrocatecol, which generates a blue-black product; orthophenylene diamine (OPD) and 3,3′,5,5′-tetramethyl benzidine hydrochloride (TMB);
(ii) alkaline phosphatase (AP) and para-Nitrophenyl phosphate, naphthol AS-MX phosphate, Fast Red TR and Fast Blue BB, napthol AS-BI phosphate, napthol AS-TR phosphate, 5-bromo-4-chloro-3-indoxyl phosphate (BCIP), Fast Red LB, Fast Garnet GBC, Nitro Blue Tetrazolium (NBT), and iodonitrotetrazolium violet (INT); and
(iii) β-D-galactosidase (β-D-Gal) with a chromogenic substrate (e.g., p-β-D-galactosidase) or fluorogenic substrate (e.g., 4-methylumbelliferyl-β-D-galactosidase).

In one embodiment, the label or reporter molecule may be selected in the group consisting of a fluorescent molecule, a radioisotope, an enzyme, a biotin, a streptavidin. For example, a label may be a fluorescent molecule.

In some embodiments, the predetermined signal may be a fluorescent signal.

The quantification may be a geometric mean of fluorescence intensity.

The quantification may be a ratio of a measured predetermined signal from an isolated sample to be tested over a measured predetermined signal from a control sample, e.g., from engineered human glomerular endothelial cells in absence of non-HLA antibodies.

In some embodiments, a quantification obtained at step b) may be obtained by (i) measuring the geometric mean fluorescence intensity of engineered human glomerular endothelial cells in presence of a blood sample from a patient presumed to contain non-HLA antibodies (Geo MFI sample), (ii) measuring the geometric mean fluorescence intensity of engineered human glomerular endothelial cells in absence of non-HLA antibodies, obtained for example by incubating the cells in a buffer such as PBS (Geo MFI control), and (iii) computing the ratio Geo MFI sample/Geo MFI control. The fluorescence signal may be obtained by contacting the cells previously contacted with the blood sample or the buffer with a secondary antibody labelled with a fluorescent probe, as above indicated.

The predetermined reference value of step c) may be obtained in a similar manner but with a blood sample obtained from patient known to have non-HLA antibodies. The predetermined reference value of step c) may be a mean of ratios thus obtained.

In some embodiments, the predetermined reference value of step c) may be obtained by quantification of a predetermined signal measured from engineered human glomerular endothelial cells as disclosed herein, or obtained according to the method as disclosed herein, in absence of non-HLA antibodies. A predetermined signal may be as above-disclosed.

For obtaining a predetermined reference value, the cells may be incubated in a medium not containing any non-HLA antibodies, such as a serum of a healthy volunteer, or alternatively not containing any antibodies, such as a buffer. A suitable buffer may any physiologically acceptable buffer such as a phosphate buffer, a HEPES buffer, or a citrate buffer.

The predetermined signal which is measured for the predetermined reference value is preferably of the same nature than the signal measured to quantify non-HLA antibodies from a blood sample of an individual to be tested which are bound to the cells. For example, the signal may be a fluorescent, luminescent, radioactive, or colorimetric signal as previously detailed.

In some embodiments, a predetermined reference value of step c) may be obtained by quantifying a signal obtained from engineered human glomerular endothelial cells incubated in a media not containing any antibodies such as a buffer. Any suitable buffer may be used, such as a phosphate buffer.

To obtain a predetermined signal of same nature than the signal measured to quantify non-HLA antibodies from a blood sample of an individual to be tested, the cells are submitted to the same procedure but in absence of any non-HLA antibodies.

For example, when the signal to be quantified is obtained from labelled secondary antibodies, the cells may be contacted with the labelled secondary antibodies in absence of any non-HLA antibodies, and then washed, and the quantification of the signal corresponding to the label of the secondary antibodies is obtained.

In some embodiments, the predetermined reference value of step c) may be obtained by incubating at least an engineered human glomerular endothelial cell as disclosed herein, or obtained according to a method as disclosed herein, with at least a blood sample of an individual known to not contain non-HLA antibodies and quantification of antibodies bound to said engineered human glomerular endothelial cell.

In some embodiments, the predetermined reference value of step c) may be obtained by quantification of antibodies bound to at least an engineered human glomerular endothelial cell as disclosed herein or obtained according to the method as disclosed herein, said antibodies being from a blood sample of an individual known to not contain non-HLA antibodies.

A blood sample of an individual known to not contain non-HLA antibodies is a control or reference blood sample. It may be a blood sample from a healthy volunteer. Such blood sample is presumed to contain antibodies other than non-HLA antibodies. Instead of a blood sample, one may use a pool serum of healthy volunteers. A “healthy volunteer” according to the invention, is an individual whose physiological state does not require a kidney transplant. Individuals who suffer from a disease which may require a kidney transplant are not considered as healthy volunteers according to the invention.

Alternatively, in some embodiments, the predetermined reference value of step c) may be obtained by quantification of antibodies bound to at least an engineered human glomerular endothelial cell as disclosed herein or obtained according to the method as disclosed herein, said antibodies being from a blood sample of an individual known to contain non-HLA antibodies.

In some embodiments, the predetermined reference value of step c) may be a ratio of a first and of second quantifications of antibodies.

A first quantification may be a quantification of antibodies bound to an engineered glomerular endothelial cell as disclosed herein, the antibodies being from a blood sample of an individual known to contain non-HLA antibodies.

A first quantification may be a quantification of a predetermined signal obtained from antibodies bound to an engineered glomerular endothelial cell as disclosed herein, the antibodies being from a blood sample of an individual known to contain non-HLA antibodies.

A first quantification may be a mean obtained from a group of individuals known to have non-HLA antibodies.

A second quantification may be a quantification measured from engineered human glomerular endothelial cells as disclosed herein or obtained according to the method as disclosed herein, in absence of non-HLA antibodies.

A second quantification may be a quantification of a predetermined signal obtained from engineered human glomerular endothelial cells as disclosed herein or obtained according to the method as disclosed herein, in absence of non-HLA antibodies.

Alternatively, a second quantification may be a quantification of antibodies bound to an engineered glomerular endothelial cell as disclosed herein, the antibodies being from a blood sample of an individual known to not contain non-HLA antibodies.

Alternatively, a second quantification may be a quantification of a predetermined signal obtained from antibodies bound to an engineered glomerular endothelial cell as disclosed herein, the antibodies being from a blood sample of an individual known to not contain non-HLA antibodies.

In some embodiments, the predetermined reference value of step c) may be a ratio of quantifications, said ratio being equal to a first quantification of a predetermined signal obtained from antibodies bound to an engineered glomerular endothelial cell as disclosed herein, said antibodies being from a blood sample of an individual presumed to contain non-HLA antibodies, over a second quantification of a predetermined signal obtained from engineered human glomerular endothelial cells as disclosed herein, or obtained according to the method as disclosed herein, in absence of non-HLA antibodies.

In some embodiments, the predetermined reference value of step c) is a ratio of a quantification of antibodies bound to an engineered glomerular endothelial cell as disclosed herein, or obtained according to the method as disclosed herein, said antibodies being from a blood sample of an individual known to contain non-HLA antibodies over a quantification of antibodies bound to an engineered glomerular endothelial cell as disclosed herein, or obtained according to the method as disclosed herein, in absence of non-HLA antibodies.

In some embodiments, the predetermined reference value of step c) may be a ratio of a first quantification of antibodies bound to an engineered glomerular endothelial cell as disclosed herein, said antibodies being from a blood sample of an individual known to contain non-HLA antibodies over a second quantification of antibodies bound to an engineered glomerular endothelial cell as disclosed herein, or obtained according to the method as disclosed herein, said antibodies being from a blood sample of an individual known to not contain non-HLA antibodies.

The quantification of the antibodies may be carried out by determining a predetermined signal.

A determination of a predetermined signal may be carried out as above indicated. A predetermined signal may be any type of suitably detectable signal as above indicated, such as a fluorescent, luminescent, radioactive, or colorimetric signal.

In some embodiments, the predetermined signal may be a fluorescent signal.

The quantification may be a geometric mean of fluorescence intensity.

The quantification of antibodies bound to an engineered glomerular endothelial cell according as disclosed herein may be a geometric mean of fluorescence intensity.

A ratio of quantifications may be a ratio of geometric means of fluorescence intensity.

A ratio of quantifications of antibodies bound to an engineered glomerular endothelial cell as disclosed herein may be a ratio of geometric means of fluorescence intensity.

A ratio of quantifications may be a ratio of first geometric mean of fluorescence intensity obtained from a first quantification obtained from antibodies bound to an engineered glomerular endothelial cell as disclosed herein over a second geometric mean of fluorescence intensity obtained from a second quantification obtained from engineered glomerular endothelial cell as disclosed herein in absence of non-HLA antibodies.

A ratio of quantifications may be a ratio of first geometric mean of fluorescence intensity obtained from a first quantification obtained from antibodies bound to an engineered glomerular endothelial cell as disclosed herein, said antibodies being from a blood sample of an individual known to contain non-HLA antibodies, over a second geometric mean of fluorescence intensity obtained from a second quantification obtained from engineered glomerular endothelial cell as disclosed herein, in absence of non-HLA antibodies.

A ratio of quantifications of antibodies bound to an engineered glomerular endothelial cell as disclosed herein may be a ratio of first geometric mean of fluorescence intensity obtained from a first quantification obtained from antibodies bound to an engineered glomerular endothelial cell as disclosed herein over a second geometric mean of fluorescence intensity obtained from a second quantification obtained from antibodies bound to an engineered glomerular endothelial cell as disclosed herein, in absence of non-HLA antibodies.

The first and second quantifications may be as disclosed above.

A predetermined value of reference at step c) may a ratio of geometric mean of fluorescence. A ratio of geometric means of fluorescence intensity may be within a range from about 1.20 to about 2.20, or from about 1.40 to about 2.00, or from about 1.50 to about 1.90, or from about 1.60 to about 1.80. For example, the ratio may be about 1.87.

In some embodiments, a ratio of geometric means of fluorescence intensity may be within a range from about 1.20 to about 3.50, or from about 1.20 to about 3.20, or from about 1.20 to about 3.00, or from 1.20 to about 2.80, or from about 1.30 to about 2.20, or from about 1.40 to about 2.10, from about 1.50 to about 2.00, or from about 1.50 to about 1.90, or from about 1.60 to about 1.80. For example, the ratio may be about 1.87 or about 2.50.

In some embodiments, the cells used in the diagnostic methods disclosed herein may be in suspension.

In some embodiments, the cells used in the diagnostic methods disclosed herein may be adhered to a support, for example in a multi-wells plate.

The cells may be suspension or adhered to a support bathed in a physiologically acceptable buffer or a suitable cell culture medium. A physiologically acceptable buffer may be buffer as above indicated, and for example may be a phosphate buffer. A physiologically acceptable buffer may comprise a chelating agent as above indicated, and for example such as EDTA, an isotonic agent, such as sucrose, and/or a blocking protein, as above indicated, and for example such as BSA.

A suitable buffer may be a phosphate buffer comprising BSA and EDTA.

In some embodiments, a ratio of a quantification described herein for step c) may be used as a biomarker for determining a likelihood of occurrence of a non-HLA antibody mediated rejection against a renal allograft in an individual in need thereof.

In some embodiments, the invention relates to a ratio of quantifications, said ratio being equal to a ratio of a first quantification of a predetermined signal obtained from antibodies bound to an engineered glomerular endothelial cell as disclosed herein, said antibodies being from a blood sample of an individual presumed to contain non-HLA antibodies, over a second quantification of a predetermined signal obtained from engineered human glomerular endothelial cells as disclosed herein, or obtained according to the method as disclosed herein, in absence of non-HLA antibodies, as a biomarker for use in a method for determining the likelihood of occurrence of a non-HLA antibody mediated rejection against a renal allograft in an individual in need thereof.

In some embodiments, the invention relates to a ratio of a first quantification of antibodies bound to an engineered glomerular endothelial cell comprising a reduction in expression of a human leukocyte antigen (HLA), said antibodies being from a blood sample of an individual known to contain non-HLA antibodies over a second quantification of antibodies bound to an engineered glomerular endothelial cell comprising a reduction in expression of a human leukocyte antigen (HLA), or obtained according to the method as disclosed herein, said antibodies being from a blood sample of an individual known to not contain non-HLA antibodies, as a biomarker for use in a method for determining the likelihood of occurrence of a non-HLA antibody mediated rejection against a renal allograft in an individual in need thereof.

In some embodiments, the invention relates to a ratio of a first quantification of antibodies bound to an engineered glomerular endothelial cell comprising a reduction in expression of a human leukocyte antigen (HLA), said antibodies being from a blood sample of an individual known to contain non-HLA antibodies over a second quantification of antibodies bound to an engineered glomerular endothelial cell comprising a reduction in expression of a human leukocyte antigen (HLA), or obtained according to the method as disclosed herein, in absence of non-HLA antibodies, as a biomarker for use in a method for determining the likelihood of occurrence of a non-HLA antibody mediated rejection against a renal allograft in an individual in need thereof.

In step c), the quantification obtained at step b) of the method disclosed herein may be expressed as a ratio of the measured predetermined signal of non-HLA antibodies bound to engineered human glomerular endothelial cells over a measured predetermined signal obtained from engineered human glomerular endothelial cells in absence of non-HLA antibodies. This ratio is then compared with a predetermined reference value as above disclosed.

In some embodiments, an in vitro method of the invention may further comprise a step of determining the likelihood of occurrence of an anti-HLA antibody mediated rejection against a renal allograft.

In some embodiments, an in vitro method of the invention may be a method for determining the likelihood of occurrence of a renal allograft rejection in an individual in need thereof, said method comprising at least the steps of:
(i) determining the likelihood of occurrence of a non-HLA antibody mediated rejection against a renal allograft according to a method as disclosed herein, and
(ii) determining the likelihood of occurrence of an anti-HLA antibody mediated rejection against a renal allograft.

Steps (i) and (ii) may be carried in any sequence of order. Step (i) may carried before, after or in parallel with step (ii).

The determination of likelihood of occurrence of an anti-HLA antibody mediated rejection against a renal allograft may be carried by any methods known in the field, for example as disclosed Tait 2016 (Frontiers in Immunology, 2016, 7: 570). Suitable methods may include Complement-Dependent Cytotoxicity, Flow Cytometry, Solid Phase Antibody Detection Assays, such as Enzyme-Linked Immunosorbent Assay or a Luminex® Bead Technology.

For example, anti-HLA antibodies may be detected using a Luminex platform applying LABScreen kits (One Lambda, Canoga Park, CA, USA) according to manufacturer's protocol. Alternatively, anti-HLA antibodies may be detected as disclosed in Picascia et al. (Clin Exp Nephrol. 2012 June; 16(3):373-81). Also, anti-HLA antibodies may be detected by ELISA or complement-dependent cytotoxicity as disclosed in Christiaans et al. (Transplantation: Mar. 15, 2000—Volume 69—Issue 5—p 917-927).

The likelihood of occurrence of an anti-HLA antibody mediated rejection against a renal allograft may be determined by a complement-dependent cytotoxicity, flow cytometry, enzyme-linked immunosorbent assay, or by Luminex® Bead Technology.

In a complement-dependent cytotoxicity assay a blood sample of an individual in need thereof is incubated in presence of potential donor lymphocytes using rabbit serum as a source of complement. If HLA-DSA are present lysis of the cells occurs. This lysis can be detected by the method of dye exclusion or by fluorescence.

In a flow cytometry assay, donor cells are incubated with a blood sample of an individual in need thereof, and then a fluorescein-labeled second anti-human immunoglobulin antibody adding which can bind to the individual's antibodies bound to the donor cells, if present.

In an enzyme-linked immunosorbent assay, HLA molecules are adsorbed in wells of microtiter trays, and a blood sample of an individual in need thereof is added. After washing of the wells, a secondary antibody, e.g., an anti-human IgG labeled with a reporter molecule, for example as above indicated, is then added. The secondary antibody will bind to the anti-HLA antibody if present.

The Luminex® Bead Technology consists in the use of beads impregnated with differing ratios of two fluorochromes resulting in a unique signal for each bead and which have one or several types of HLA molecules attached. A blood sample of an individual in need thereof is incubated. If HLA antibodies are present, they will react with the bead expressing the appropriate HLA molecule. After washing, the beads are incubated with a secondary antibody labeled with a reporter molecule.

According to another of its objects, the invention relates to a method for determining the likelihood of occurrence of a non-HLA antibody mediated rejection against a renal allograft in an individual in need thereof and administering a treatment against a non-HLA antibody mediated rejection against a renal allograft in said individual in need thereof, the method comprising at least the steps of:
i) carrying out the in vitro diagnostic method as disclosed herein, and
ii) administering to said individual a treatment for preventing and/or reducing a non-HLA antibody mediated rejection against a renal allograft if said patient is determined to be at risk of a non-HLA antibody mediated rejection against a renal allograft.

An appropriate therapeutic treatment as referred to above can be chosen from any known treatment currently available and which is usually prescribed to an individual who is at risk for or who suffers from antibody-mediated rejection.

Such treatments are well known to one skilled in the art and include, but are not limited to treatments comprising immunosuppressant drugs, plasma exchanges; immuno-adsorptions; intravenous immune globulins; or drugs targeting antibodies, B lymphocytes or plasma cells depleting agents.

As example of drugs targeting antibodies, one may mention imlifidase. As example of drugs targeting B lymphocytes, one may mention anti-CD20 monoclonal antibodies, such as rituximab. And as example of drugs targeting plasma cells, one may mention anti-CD38 monoclonal antibodies, such as bortezomib.

The different treatments indicated above may be combined in a patient to optimize the outcomes.

Plasma exchange and intravenous immune globulins, with or without rituximab, is the most commonly used strategy and is generally considered standard of care for antibody-mediated rejection treatment.

### Kits

The in vitro methods and kits described herein may also be implemented as “companion tests” to improve diagnostic methods and to improve methods of treatment regularly used to cure or prevent acute organ rejection in an individual before or after a renal allograft.

According to another of its objects, the invention relates to a kit for determining the likelihood of occurrence of a non-HLA antibody mediated rejection against a renal allograft in an individual in need thereof, the kit comprising:
(i) at least one engineered human glomerular endothelial cell as disclosed herein, and
(ii) at least one mean to detect and quantify antibodies bound on said human glomerular endothelial cell.

The kit as disclosed herein may further comprise at least one instruction to implement an in vitro diagnostic method as disclosed.

The kit as disclosed herein may further comprise an instruction to compare a quantification of a predetermined signal of non-HLA antibodies bound to said engineered human glomerular endothelial cell with a predetermined reference value, wherein the predetermined reference value is a ratio of a first quantification of a predetermined signal obtained from antibodies bound to an engineered glomerular endothelial cell comprising a reduction in expression of a human leukocyte antigen (HLA), said antibodies being from a blood sample of an individual known to contain non-HLA antibodies over a second quantification of a predetermined signal obtained from an engineered glomerular endothelial cell comprising a reduction in expression of a human leukocyte antigen (HLA), in absence of non-HLA antibodies.

The mean to detect and quantify antibodies bound on said human glomerular endothelial cell may be as above described and may be for example a labeled anti-human immunoglobulin antibody, or a fragment thereof.

A kit as disclosed herein may comprise a buffer as above disclosed and a secondary antibody linked to a reporter molecule as above indicated, such as a fluorescent molecule, and able to bind the non-HLA antibodies.

A kit of the invention may comprise one container containing the engineered human glomerular endothelial cell as disclosed herein and one container containing at least one mean to detect and quantify antibodies bound on said human glomerular endothelial cell.

A kit of the invention may comprise one or more other containers, containing for example, wash reagents or buffers.

In a kit of the invention, the engineered human glomerular endothelial cells may be stored in a frozen state.

A kit may comprise means for acquiring a quantity of a blood or serum sample; wash reagents and buffers, engineered human glomerular endothelial cells as disclosed herein, and means to detect and quantify antibodies bound on said human glomerular endothelial cell.

Further a kit may comprise means and reagents to isolate antibodies from an isolated blood or serum sample.

In some embodiment, the invention relates to a method for manufacturing a kit comprising a step of placing in a package a container comprising engineered human glomerular endothelial cells as disclosed herein and an instruction to compare a quantification of a predetermined signal of non-HLA antibodies bound to said engineered human glomerular endothelial cell, said non-HLA antibodies being obtained from an isolated biological sample from an individual presumed to have non-HLA antibodies, with a predetermined reference value, wherein the predetermined reference value is a ratio of a first quantification of a predetermined signal obtained from antibodies bound to an engineered glomerular endothelial cell comprising a reduction in expression of a human leukocyte antigen (HLA), said antibodies being from a blood sample of an individual known to contain non-HLA antibodies over a second quantification of a predetermined signal obtained from an engineered glomerular endothelial cell comprising a reduction in expression of a human leukocyte antigen (HLA), in absence of non-HLA antibodies.

The instruction may be printed on a leaflet and the printed leaflet may be placed in the package or the instruction may be printed on the package.

The method of manufacture of a kit of the invention may further comprise steps of preparing engineered human glomerular endothelial cells as disclosed herein. The cells may be prepared and provided in the kit as a cell suspension, cells plated on a culture plate, in a frozen state, or in a lyophilized state.

A kit may further comprise culture media for the engineered human glomerular endothelial cells as disclosed herein.

According to another of its objects, the invention relates to a use of a kit as disclosed herein for determining the likelihood of occurrence of a non-HLA antibody mediated rejection against a renal allograft in an individual in need thereof.

In some embodiments, a kit disclosed herein may be for use in a method for determining the likelihood of occurrence of a non-HLA antibody mediated rejection against a renal allograft in an individual in need thereof, the method comprising a step of comparing a quantification of a predetermined signal of non-HLA antibodies bound to the engineered human glomerular endothelial cell, the quantification being obtained as above indicated, with a predetermined reference value, wherein the predetermined reference value is a ratio of a first quantification of a predetermined signal obtained from antibodies bound to an engineered glomerular endothelial cell comprising a reduction in expression of a human leukocyte antigen (HLA), said antibodies being from a blood sample of an individual known to contain non-HLA antibodies over a second quantification of a predetermined signal obtained from an engineered glomerular endothelial cell comprising a reduction in expression of a human leukocyte antigen (HLA), in absence of non-HLA antibodies.

Some embodiments also relate to a use of a kit as disclosed herein for obtaining a quantification of a predetermined signal of non-HLA antibodies bound to engineered human glomerular endothelial cell and comparing said quantification with a predetermined reference value, wherein the predetermined reference value is a ratio of a first quantification of a predetermined signal obtained from antibodies bound to an engineered glomerular endothelial cell comprising a reduction in expression of a human leukocyte antigen (HLA), said antibodies being from a blood sample of an individual known to contain non-HLA antibodies over a second quantification of a predetermined signal obtained from an engineered glomerular endothelial cell comprising a reduction in expression of a human leukocyte antigen (HLA), in absence of non-HLA antibodies.

The in vitro methods and kits described herein may provide clinical information that may be used as such, or that may be used additionally to clinical information that is provided by known methods such as the in vitro observation of a biopsy sample previously collected from the grafted individual. Illustratively, the in vitro methods and kits described herein allow completing information relating to a biopsy sample exhibiting lesions typical from the presence of anti-endothelial cells antibodies, and especially allow completing information relating to a biopsy sample exhibiting lesions typical from the presence of non-HLA antibodies.

Illustratively, the in vitro methods and kits described herein allow determining the presence of non-HLA antibodies in an individual undergoing an acute rejection of an allograft, and especially of a renal allograft, wherein the detection of non-HLA antibodies may permit the medical practitioner to maintain or adapt the therapeutic treatment to be administered to the allografted individual. Adapting an allografted individual treatment encompasses administering to the said individual one or more active ingredients aimed at reducing or blocking the deleterious effects of non-HLA antibodies, caused to the grafted organ tissue.

Companion tests are diagnostic tests used as companion to a therapeutic drug to determine its applicability to a specific person. They are co-developed with drugs to aid in selecting or excluding patient groups for treatment with that particular drug on the basis of their biological characteristics that determine responders and non-responders to the therapy. They are developed based on companion biomarkers, biomarkers that prospectively help predict likely response or severe toxicity.

For instance, a strategy of treatment of acute microvascular rejection, or a non-HLA antibody mediated rejection against a renal allograft, including an in vitro method according to the invention as a companion test may consist in the following steps:
selecting an individual that is a renal allograft candidate;
transplanting said candidate with a kidney;
determining the likelihood of occurrence of an acute microvascular rejection (AMVR) against a renal allograft in the transplant recipient, or of a non-HLA antibody mediated rejection against a renal allograft, using an in vitro method as disclosed herein following the transplant;
treating said transplant recipient with an appropriate therapeutic treatment to avoid an acute microvascular rejection (AMVR), or a non-HLA antibody mediated rejection, against the renal allograft;
after an appropriate lapse of time has passed and the treatment has had time to have an effect on the recipient, determining once again the likelihood of occurrence of an acute microvascular rejection (AMVR), or of a non-HLA antibody mediated rejection, against a renal allograft in the transplant recipient using an in vitro method according to the invention,
comparing the likelihood of occurrence before and after treatment of the transplant recipient in order to determine whether said treatment has decreased the likelihood of occurrence of an acute microvascular rejection (AMVR), or a non-HLA antibody mediated rejection, which would suggest that the treatment has been successful.

The invention will be further understood from the following non-limiting examples. The following examples are provided to describe in detail some of the representative, presently preferred methods and materials of the invention. These examples are provided for purposes of illustration of the inventive concepts and are not intended to limit the scope of the invention as defined by the appended claims.

### EXAMPLES

### Materials & Methods

### Patients

We retrospectively considered all consecutive adult patients who received a kidney transplant at Necker Hospital (Paris, France) between January 2012 and June 2017 with available serum collected immediately before transplantation (DO) and available follow-up at our institution. Exclusion criteria included pretransplantation desensitization with plasma exchanges for ABO- or HLA-incompatibility (N=31), active infection with human immunodeficiency virus, hepatitis C or B virus (N=40), and primary nonfunction of the transplanted kidney due to surgical issues or arterial thrombosis (N=6). Ultimately, 389 KTRs were included. All participating patients provided written informed consent.

The transplantation allocation system followed the rules of the French national agency for organ procurement (Agence de la Biomédecine). Negative IgG T cell and B cell complement-dependent cytotoxicity crossmatching was required for all of the KTRs. Clinical and biological data for the donors and recipients were retrospectively obtained from the registry Données Informatiques Validées en Transplantation (DIVAT, www.divat.fr/) and the medical records of the patients.

### Histologic Assessment

Histologic assessments of all the biopsies performed during follow-up for each patient were collected. Biopsy specimens were fixed in formalin, acetic acid and alcohol and embedded in paraffin. Tissue sections were stained with hematoxylin and eosin, Masson's trichrome, periodic acid-Schiff reagent, and Jones' stain for light microscopy evaluation. C4d immunohistochemical staining was systematically performed (with a rabbit anti-human C4d monoclonal Ab; 1:200 dilution; Clinisciences, Nanterre, France).

Protocol biopsies were performed at 3 and 12 months posttransplantation, and indication biopsies were performed for clinical indications. Biopsies were classified using the Banff 2015 and 2017 updates of the Banff classification system (Haas, M. et al. American Journal of Transplantation (2018); Loupy, A. et al. Am. J. Transplant. 17, 28-41 (2017)). The biopsies were declared inadequate if the number of glomeruli was strictly below 8. The biopsies were graded from zero to three according to the Banff histologic parameters for ptc, g, t, i, ci, ct, cv, ah, and allograft glomerulopathy (cg) (Haas, M. et al. Am. J. Transplant. 14, 272-283 (2014).)

For the purpose of the study, the term “ABMRh” was used for biopsy specimens that fulfilled the first two (histologic) Banff 2015 and 2017 criteria for ABMR by combining Banff scores for g, ptc, arteritis, thrombotic microangiopathy, and C4d deposition (Senev, A. et al. Am. J. Transplant. 19, 763-780 (2019).)

### Detection of HLA-DSAs

All of the patients were tested for the presence of HLA-DSAs immediately before transplantation (DO), at 3 months, at 12 months and for clinical indications. The presence of circulating HLA-DSAs against HLA-A, HLA-B, HLA-Cw, HLA-DR, HLA-DQ, and HLA-DP was determined using single-antigen flow bead assays (One Lambda, Inc., Canoga Park, CA) on a Luminex platform. Beads with a normalized mean fluorescence intensity (MFI) greater than 1,000 arbitrary units were considered positive.

### Cell Culture

### HRECs

Normal HRECs were harvested from human nephrectomy specimens removed for renal cell carcinoma and isolated according to previously published methods, with minor modifications (Pallet, N. et al. Kidney Int. 67, 2422-33 (2005); Anglicheau, D. et al. Kidney Int. 70, 1019-25 (2006).)

Fragments of the nonmalignant renal cortex were minced and digested with collagenase IV (Roche, 250 IU/ml) for 3 h at 37° ° C. Cells were centrifuged, and the pellets were washed three times with phosphate-buffered saline (PBS). Cells were then cultured in Dulbecco's modified Eagle's medium containing 10 μg/ml human apotransferrin, 500 ng/ml hydrocortisone (Sigma-Aldrich), 10 ng/ml epidermal growth factor (Sigma-Aldrich), 6.5 ng/ml triiodothyronine (Sigma-Aldrich), 1% fetal calf serum, 25 IU/ml penicillin and 25 μg/ml streptomycin (Thermo Fisher, Courtaboeuf, France) and supplemented with insulin-transferrin-selenium (Thermo Fisher). Cells were incubated at 37° C. in 5% CO2 and 95% air. The characterization of our cellular model has been published previously (Pallet, N. et al. Kidney Int. 67, 2422-33 (2005)), confirming the proximal descent of the vast majority of the cultured tubular epithelial cells. Experiments were not performed with cells beyond the fourth passage, as it has been shown that no phenotypic changes occur up to this passage number (Detrisac, C. J., Sens, M. A., Garvin, A. J., Spicer, S. S. & Sens, D. A. Kidney Int. 25, 383-90 (1984).)

### HK-2 Cells

HK-2 cells were obtained and cultured as previously described (Amrouche, L. et al. J. Am. Soc. Nephrol. 28, 479-493 (2017).)

### CiGEnC

CiGEnC cells were kindly provided by SC. Satchell (Satchell, S. C. et al. Kidney Int. 69, 1633-1640 (2006)) and were cultured in endothelial growth medium 2-microvascular (EGM2-MV; Promocell, Heideberg, Germany) in culture flasks previously coated with 0.1% gelatin (Sigma-Aldrich). CiGEnC proliferated at 33ºC, with growth arrest and differentiation occurring after culture at 37ºC for 7 days (Satchell, S. C. et al. Kidney Int. 69, 1633-1640 (2006)).

### CRISPR/Cas9 Genome Editing

gRNA sites in B2M and CIITA exonic loci were identified using the online optimized design software (Haeussler, M. et al. Genome Biol. 17, 148 (2016)) at crispor.tefor.net. The highest scoring gRNAs, which had no off-target sequences with perfect matches in the human genome, and the nearest coding off-target exonic sites containing at least 3 mismatched nucleotides were selected and purchased from Thermo Fisher (TrueGuide 2-piece modified Synthetic gRNA, Thermo Fisher). The B2M crispr RNA (crRNA)-targeting sequences included GAGTAGCGCGAGCACAGCTA (B2M exon 1) and AGTCACATGGTTCACACGGC (B2M exon 2). The CIITA crRNAs targeting sequences included CATCGCTGTTAAGAAGCTCC exon (CIITA 2) and GATATTGGCATAAGCCTCCC (CIITA exon 3). crRNAs and transactivating crispr RNA (tracrRNA) were annealed in TE buffer in a Verity thermocycler (Thermo Fisher) according to the manufacturer's instructions to obtain complete functional gRNAs.

### Transfection

A Cas9 nuclease/gRNA/transfection reagent complex was prepared according to the manufacturer's instructions. Briefly, a mixture containing Cas9 nuclease (TrueCut V2, Thermo Fisher), cr-tracrRNAs, Opti MEM medium and Cas9 Plus reagent was combined with Lipofectamine™ CRISPRMAX™ Reagent (Thermo Fisher). This complex was plated on 6-well plates, and cells were added and incubated for 2 days. The cells were washed and cultured for 5 more days.

### Single-Cell Isolation of CRISPR/Cas9-Modified Cells

After transfection, CiGEnC were stimulated with IFN-γ (100 IU/ml, Miltenyi Biotec, Paris, France) for 2 days to upregulate HLA-I and HLA-II. Cells were harvested with trypsin and subsequently stained with Fixable Viability Dye eFluor 660 (Thermo Fisher) and directly conjugated VioBlue anti-HLA-ABC (Miltenyi Biotec) and BV605 anti-HLA-DR (Ozyme, Montigny-le-bretonneux, France) monoclonal Abs (mAbs).

B2M and CIITA loss-of-function were identified by live cells that did not show increased cell-surface expression of HLA-ABC or HLA-DR, respectively, with the positive threshold defined by unmodified CiGEnC stained with the same mAbs. These gates were then used to collect CiGEnCΔHLA cells using a 100-μm low-pressure nozzle on a BD FACSAria II (BD Biosciences, Le Pont de Claix, France) and then to deposit single cells into flat-bottom 96-well cell culture plates containing EGM-2/50% fetal bovine serum (FBS) medium. After 24 h, the cells were refed with fresh EGM-2/5% FBS medium that was changed every other day. Colonies were scored after 14 days and serially expanded into larger vessel sizes. To analyze phenotypic stability, CiGEnCΔHLA and unmodified CiGEnC were separately expanded in EGM-2/5% FBS for 2 weeks. These cells were then challenged with TNF-α (100 IU/ml, Miltenyi Biotec) and IFN-γ (100 IU/ml, Miltenyi Biotec) and harvested at 24 h for RT-qPCR analysis and at 48 h for both FACS analysis and immunofluorescence staining.

### PCR and Sanger Sequencing

Genomic DNA was isolated from clonally expanded CiGEnC using the QIAamp DNA Mini Kit (Qiagen, Courtaboeuf, France) according to the manufacturer's protocol. One hundred- to 150-bp segments containing the B2M and CIITA gRNA target sites were amplified by PCR using AmpliTaq Gold 360 DNA Polymerase (Thermo Fisher) using the primers B2M Exon 1 Forward (ATATAAGTGGAGGCGTCGCG), B2M Exon 1 Reverse (TGGAGAGACTCACGCTGGAT), B2M Exon 2 Forward (TGTCTTTCAGCAAGGACTGGT), B2M Exon 2 Reverse (ACCCCACTTAACTATCTTGGGC), B2M Exon 2 Forward (CTGCCTCTTTCCAACACCCT), CIITA Exon 2 Reverse (CTTCTCCAGCCAGGTCCATC), CIITA Exon 3 Forward (TTTCAGCAGGCTGTTGTGTG), and CIITA Exon 3 Reverse (GCAGCAAAGAACTCTTGCCC). The PCR amplicons were then purified and submitted for Sanger sequencing using an ABI 3730XL DNA sequencer (Eurofins Genomics, Ebersberg, Germany). Unmodified CiGEnC cells were used as a control for comparison.

The highest scoring off-target site of B2M exon 2 was TGAGAGTACCAGGTGTGACG (HDHD1P2, 4 mismatches 2:5:12:18). To determine if this site was mutated, the following sequencing primers were used: HDHD1P2_F (TCGTCGGCAGCGTCGTGCAGTCTGGGATTTGGGA) and HDHD1P2_R (GAGGGCCGTCTCGTGGGCTCGGTATGAGTGAGAGG).

To assess off-target cleavage of the highest scoring off-target coding site of CIITA exon 2 (JMJD4, CATCACTGCTAGGAAGCTTCAGG, 4 mismatches in 5:9:12:19), the following sequencing primers were used: JMJD4_F (TCGTCGGCAGCGTCATCAAAGGCTGCCTGTTCGA) and JMJD4_R (GTCTCGTGGGCTCGGTGCTCGGGCATCAACTTTGA). To assess off-target cleavage of the highest scoring off-target coding site of CIITA exon 3 (OSTF1, GATATCTGCATAACCCTTCCAGG, 4 mismatches in 6:7:14:17), the following sequencing primers were used: OSTF1_F (TCGTCGGCAGCGTCGGGAGATACAGCTTTGCATGC) and OSTF1_R (AAGACCAGTCTCGTGGGCTCGGTTCAGGGCAAGCA). Analyses of sequencing results were performed using Serial Cloner 2.6.1 software (serialbasics.free.fr/Serial_Cloner.html). Allelic analysis was performed using CRISPR-ID 1.1a 34 (crispid.gbiomed.kuleuven.be/), and the efficacy of gene editing was assessed by Tracking of Indels by Decomposition (TIDE) 35 web applications (tide.deskgen.com).

### FACS Analysis

After one week of differentiation induced by culture at 37° C., cells were stained with VioBlue-conjugated anti-HLA-ABC, PE-conjugated anti-VEGFR2, PE-Vio770-conjugated anti-ICAM2, APC-conjugated anti-VE-cadherin, APC-Vio770-conjugated anti-Tie2 (all from Miltenyi Biotec) and BV605-conjugated anti-HLA-DR (Ozyme) Abs and analyzed on an LSR Fortessa flow cytometer (BD Biosciences, San Jose, CA) with postacquisition analysis using Kaluza software (Kaluza 2.1, Beckman Coulter). To induce HLA antigen expression, cells were exposed to IFN-γ and TNF-α (100 U/mL, Miltenyi Biotec) for 48 h before staining. The RFI was calculated by subtracting the MFI of the corresponding isotype control.

gRT-PCR

RNA was isolated using the RNeasy Mini Kit (Qiagen, Courtaboeuf, France) and used to generate cDNA using a mixture containing an RNAse inhibitor, dNTP mix, Random Hexamer, MgCl2 Solution and MultiScribe Reverse Transcriptase (all from Thermo Fisher). qPCR reactions were assembled with TaqMan 2× Fast Univ. PCR Master Mix (Thermo Fisher) and predeveloped TaqMan gene expression probes and analyzed on a Viaa7 Real-Time system using QuantStudio Real-Time PCR software (Thermo Fisher). The probes used in this study were purchased from Thermo Fisher: CIITA (Hs00172106_m1), KDR (Hs00911700_m1), ICAM2 (Hs00609563_m1), CDH5 (Hs00901465_m1), TIE2 (Hs00945150_m1), B2M (Hs00187842_m1), and HLA-DR (Hs00219575_m1). For GAPDH, the following primers and probes were used: sense: 5′-CCACATCGCTCAGACACCAT-3′, antisense: 5′-TGACCAGGCGCCCAATA-3′, and probe: 5′-FAM-AGTCAACGGATTTGGTC-MGB-3′. For CXCL10, the following primers and probes were used: sense: 5′-TGTCCACGTGTTGAGATCATTG-3′, antisense: 5′-GGCCTTCGATTCTGGATTCA-3′, and probe: 5′-FAM-TACAATGAAAAAGAAGGGTGAGAA-MGB-3′. Gene expression levels were normalized to those of GAPDH. When indicated, cells were exposed to IFN-γ and TNF-α (100 U/mL) for 24 h before RNA extraction.

### Immunofluorescence

Cells grown to confluence on gelatin-coated glass coverslips were fixed in 4% formaldehyde and permeabilized in 0.1% Triton X-100 containing 3% BSA (Thermo Fisher). The cells were then incubated for 12 h at 4° C. with VioBright-515-conjugated anti-PECAM1 or APC-conjugated anti-VE-cadherin (Miltenyi Biotec) primary antibodies or unconjugated anti-HLA A/B/C, anti-HLA-DP/DR/DQ (Ozyme) or anti ICAM2 (Abcam) primary antibodies. Unconjugated primary antibody binding was detected using an Alexa Fluor 647-conjugated anti-mouse IgG secondary antibody (Ozyme). Negative controls were either isotypes for the fluorophore-conjugated primary antibodies or the absence of the primary antibody for secondary revelation. The cells were then stained with DAPI, and coverslips were mounted using Fluoromount™ (Sigma-Aldrich); the cells were examined using a Zeiss confocal microscope (Zeiss Confocal LSM 700). Zen900 software was used to generate images, and ImageJ (Java) was used to analyze the images.

### Phase-Contrast Microscopy

Unmodified CiGEnC or CiGEnCΔHLA cells at various passages were seeded in flasks at a subconfluent density and placed at either 33ºC or 37ºC, and morphology was examined by phase-contrast microscopy.

### Cell Proliferation Assay

Unmodified CiGEnC or CiGEnCΔHLA cells were seeded at 50,000 cells per well in 12-well plates, and real-time evaluation of cell confluence was performed using the IncuCyte Live Cell Imaging System (Essen BioScience, Hertfordshire, United Kingdom). Images were acquired every 2 h for 60 h from nine separate regions per well using a 10× objective and analyzed by IncuCyte basic software.

HLA-A2 CAR-T Cell Generation and an xCELLigence Cytotoxicity Assay

Healthy donor peripheral blood mononuclear cells (PBMCs) were obtained from the Etablissement Français du Sang. PBMCs were typed based on the expression or absence of HLA-A2/A28 molecules, as assessed by anti-HLA-A2/A28 antibody (OneLambda) staining evaluated by FACS analysis. CD8+ T cells were sorted using a FACSAria II (BD Biosciences), transduced 2 days after activation with an HLA-A2-specific CAR construct at a multiplicity of infection (MOI) of 40 and incubated for 18 h for transduction as previously described 36. On day 5 posttransduction, CD8+ EGFRt+ cells were sorted using a FACSAria II. CD8+ CAR-T cells were cultured in X-VIVO® 20 medium containing 10% human serum AB (Biowest) supplemented with IL-2 (100 UI/ml). For the cytotoxic assay, 1×10⁴ unmodified CiGEnC or CiGEnCΔHLA cells were seeded in E-plate (ACEA Biosciences) wells. After 15 h, 2×10⁴ CD8+ anti-HLA-A2 CAR-T cells were added to the culture. To evaluate the viability of unmodified CiGEnC or CiGEnCΔHLA cells, electrical impedance measurements were taken with an xCELLigence RTCA MP instrument (ACEA Biosciences) every 15 minutes for 10 h. The cell indices (CIs) were normalized to the reference value (measured just prior to adding CAR-T cells to the culture). The normalized cell index of experimental wells was normalized to the cell index of control wells containing only the corresponding endothelial cell line.

### Non-HLA Antibody Detection Assay (NHADIA)

Serum samples collected immediately before transplantation were tested with the NHADIA. After washing with PBS, differentiated CiGEnCΔHLA cells were trypsinized (TrypLE Express, Thermo Fisher) and washed before incubation with a fixable viability dye (Thermo Fisher) for 20 minutes at 4ºC. Then, the CiGEnCΔHLA cells were incubated with patient sera diluted 1:2 in PBS containing 0.05% BSA and 2 mM EDTA for 30 minutes. For the negative control, cells were incubated with PBS only. After two more washes, the cells were incubated with an Alexa Fluor® 488-conjugated anti-human IgG Ab (AffiniPure F(ab′)₂ Fragment Donkey Anti-Human IgG (H+L), Interchim) for 20 minutes. Fluorescence was measured by flow cytometry (FACSCanto II or LSR Fortessa X-20, BD Biosciences), and geometric means of fluorescence intensity (Geo MFIs) were calculated using Kaluza software v2.1 (Beckman Coulter, Villepinte, France). The results were calculated as the ratio of the Geo MFIₛₐₘₚₗₑ to the Geo MFI_{negativecontrol}.

### Statistical Methods

Continuous variables are described as the mean±standard deviation (SD) or median and IQR. Frequencies of categorical variables are presented as numbers and percentages. We compared continuous variables using the Mann-Whitney test or Student's t test and the proportion of categorical variables using Fisher's exact test or a chi-2 test when appropriate. P-values≤0.05 were regarded as statistically significant.

Univariate and multivariate regression analyses were performed to identify pretransplant determinants of the NHADIA. To identify the positivity threshold of the NHADIA that would best predict ABMRh independently of HLA-DSAs, time to ABMRh diagnosis was assessed in a Cox regression analysis including various thresholds of the NHADIA and HLA-DSA status at the time of transplantation. Cox proportional hazard analysis was used to associate NHADIA results with time to ABMRh. The Kaplan-Meier method was used to estimate the cumulative incidence of ABMRh, with a timescale of years posttransplantation. With a median follow-up time after transplantation of 4.6 years, time was censored at 4 years after transplantation in all the time-to-event analyses.

Alluvial plots and dendrograms were built using the “networkD3” and “ClustOfVar” R packages, respectively. Analyses were performed with R software (R Development Core Team, R version 3.6.3 and R studio version 1.2.5033) and GraphPad PRISM® software (version 7.0a; GraphPad Software, San Diego, USA).

### Example 1: Genetic Ablation of B2M and CIITA in CiGEnC

CiGEnC express both class I and class II HLA molecules. The HLA molecule expression on the cell surface of CiGEnC to obtain a CIGEnCΔHLA cell line was suppressed as described herein after.

B2M is essential for the assembly and expression of the HLA-1 complex (Serreze, D. V, Leiter, E. H., Christianson, G. J., Greiner, D. & Roopenian, D. C. Diabetes 43, 505-9 (1994).) and CIITA is a crucial transactivator of HLA-2 (LeibundGut-Landmann, S. et al. Eur. J. Immunol. 34, 1513-25 (2004).), therefore B2M and CIITA double disruption was developed to generate CiGEnCΔHLA cells.

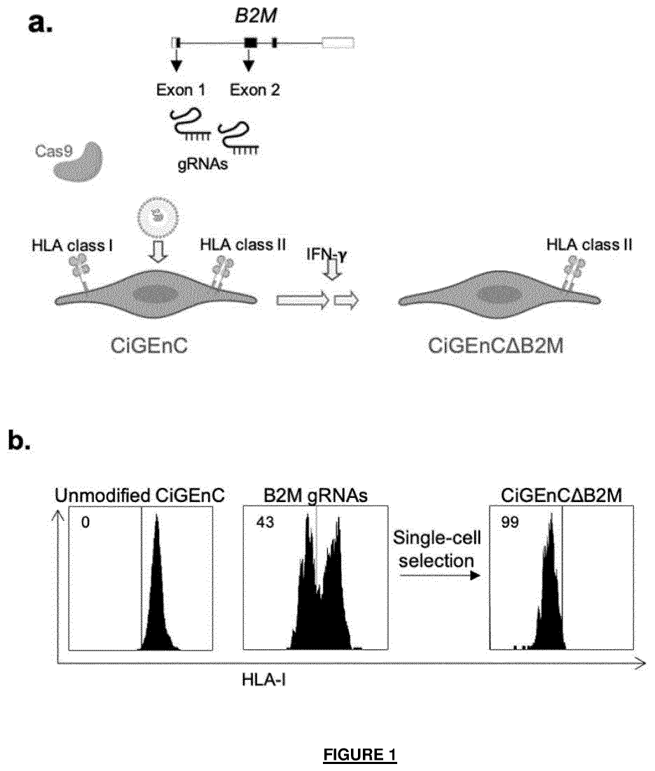

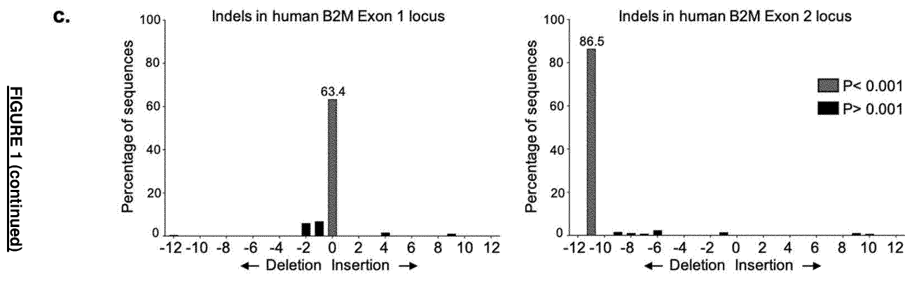

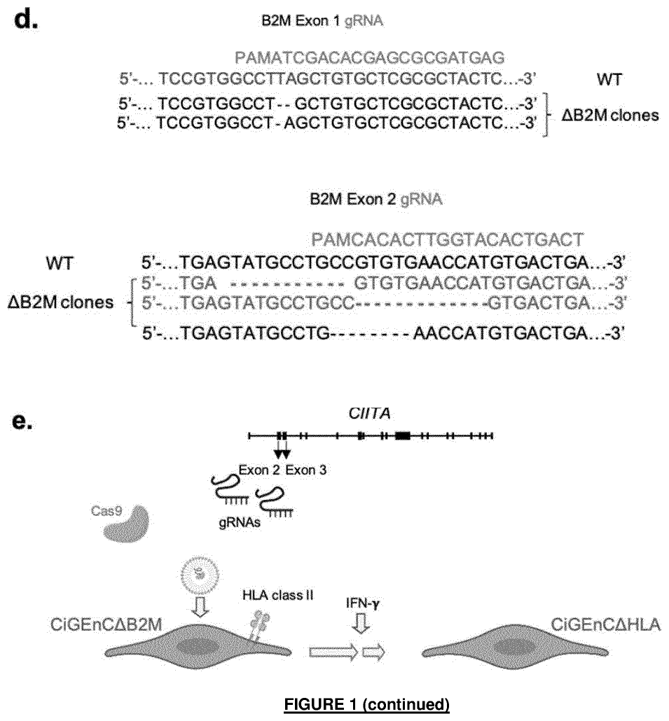

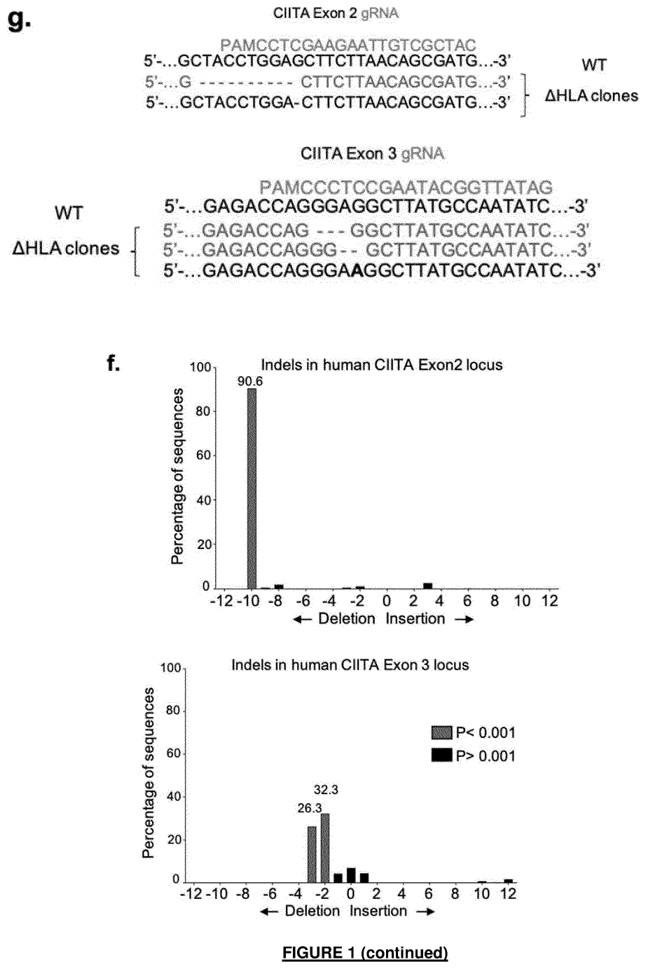

First, the ability to eliminate HLA-1 expression on CiGEnC by disrupting B2M was tested. CiGEnC at 33° C. tolerated reverse transfection of DNA with acceptable efficiency (routinely exceeding 25% after a single round of transfection, data not shown). We used Lipofectamine reagent to codeliver an active Cas9 protein and two different synthetic guide RNAs (gRNAs) targeting exonic regions (exon 1 and exon 2) shared by all known splice variants of B2M into cells, as described in the Methods and FIG. 1a and FIG. 1b.

After a single round of transfection, over 95% of the cells remained viable. B2M expression can be induced by IFN-γ, and loss-of-function identification could be simply performed through flow cytometry analysis of the surface expression of HLA-I. While >99% of unmodified CiGEnC upregulated HLA-I upon IFN-γ stimulation, delivery of B2M-specific gRNA resulted in less than 43% HLA-I-positive cells. Thus, we used single-cell FACS sorting of viable cells before clonal expansion of CiGEnCΔB2M cells (FIG. 1c).

After expansion, genomic DNA isolated from CiGEnCΔB2M clones was used to amplify the two regions containing the B2M-specific gRNA target site. Consistent with the loss-of-function results, several selected CiGEnCΔB2M clones were confirmed to have indels of between 7 and 13 bp at the predicted B2M exon 1 and/or exon 2 loci (FIG. 1d). Among these clones, we determined that one presented a biallelic deletion of 11 nucleotides in the B2M exon 2 locus, whereas no gene editing was detected in B2M exon 1 (FIG. 1d). Additionally, sequencing of the highest scoring putative off-target coding site of the B2M exon 2 locus in HDHD1P2 revealed no mutation in this clone. Of note, no off-target coding site was predicted for B2M exon 1-specific gRNA.

Having established that we can efficiently produce CiGEnC with biallelic disruption of B2M, leading to the loss of HLA-ABC expression on the cell surface (FIG. 1c), we next proceeded to disrupt CIITA (FIG. 1e).

For that purpose, we used a clone with a biallelic 11-nucleotide deletion and transfected it with two different synthetic gRNAs targeting exonic regions (exon 2 and exon 3) shared by all known splice variants of CIITA (FIG. 1g).

Similar to B2M, CIITA is also IFN-γ inducible, but loss-of-function identification by flow cytometry analysis of surface expression of HLA-DR is more complicated because only 6% of undifferentiated CiGEnC expressed HLA-DR at 33° ° C. even after cytokine stimulation (data not shown). Delivery of CIITA-specific gRNAs resulted in less than 3% HLA-DR-positive cells. Nevertheless, we used single-cell FACS sorting of viable cells before clonal expansion of CiGEnCΔHLA cells. After expansion, genomic DNA isolated from CiGEnCΔHLA clones was used to amplify the two regions containing the CIITA-specific gRNA target site as well as off-target sites. Thus, several selected CiGEnCΔHLA clones were confirmed to have indels of between 1 and 11 bp at the predicted CIITA exon 2 and/or exon 3 loci (FIG. 1f).

### Example 2: Characterization of HLA Antigen Loss-of-Expression

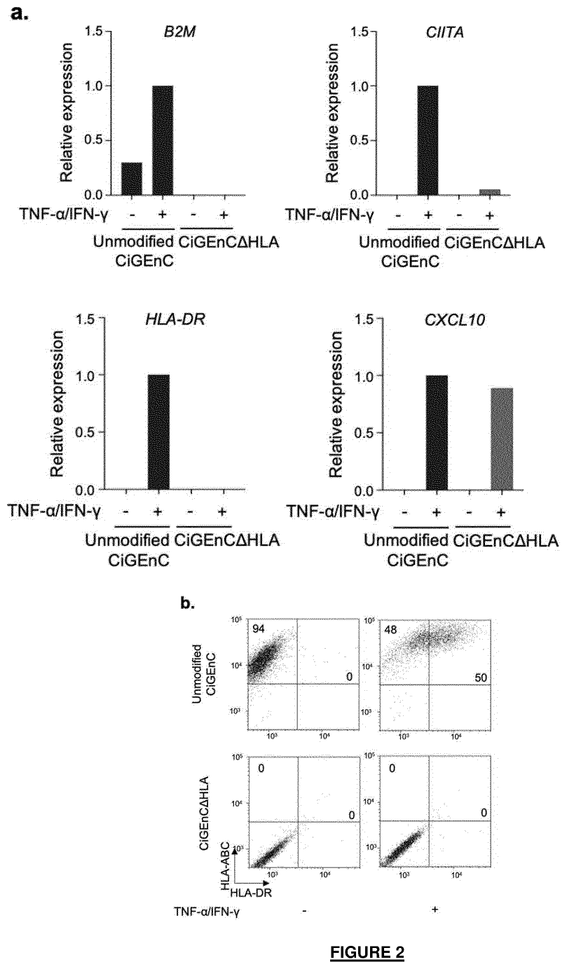

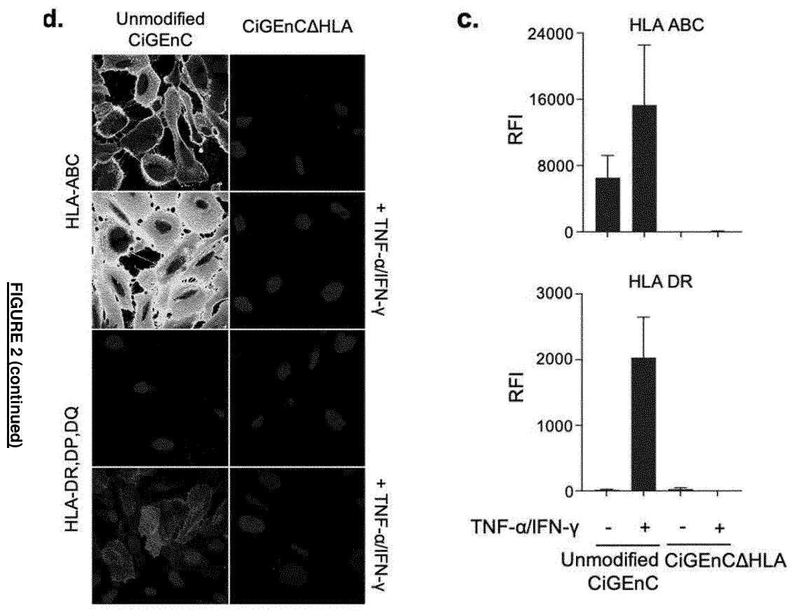

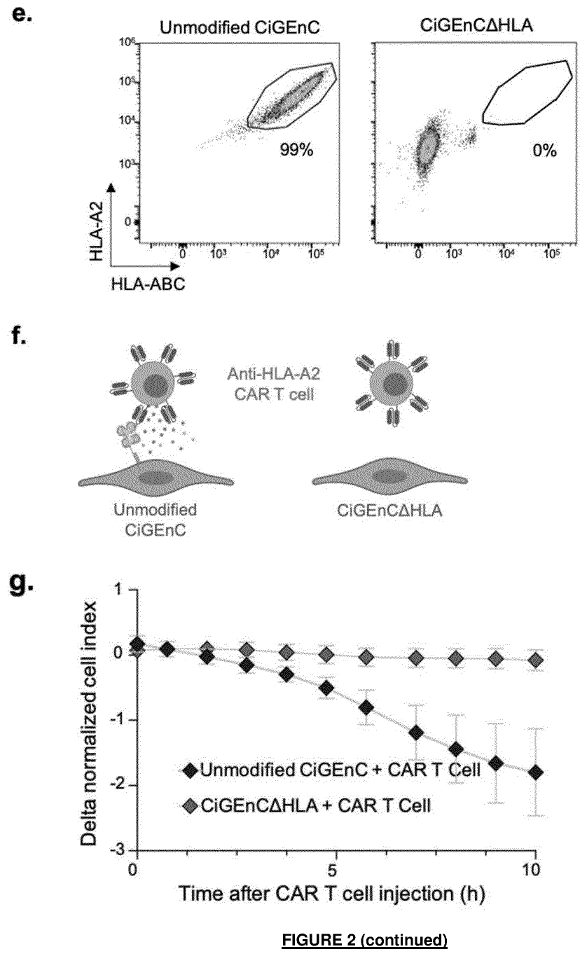

After 7 days of differentiation at 37° ° C. followed by cytokine stimulation, RT-qPCR analysis of unmodified CiGEnC revealed, as expected, basal expression of B2M that increased after cytokine stimulation and inducible expression of CIITA and HLA-DR after cytokine stimulation. In contrast, analysis of the CiGEnCΔHLA clone revealed a >99.9% reduction in B2M, 95% reduction in CIITA and undetectable HLA-DR mRNA expression but equivalent mRNA levels of CXCL10, another IFN-γ-inducible gene (FIG. 2a).

Thus, we confirmed ablation of both B2M and CIITA by monitoring the absence of HLA antigens by FACS and immunofluorescence analyses of unmodified CiGEnC and CiGEnCΔHLA cells after cytokine stimulation. As described in FIG. 2b, without cytokine stimulation, more than 94% of unmodified CiGEnC expressed HLA-ABC but exhibited very limited HLA-DR expression. When both IFN-γ and TNF-α were added to the culture for 48 h, more than 50% of unmodified CiGEnC expressed HLA-DR, as expected. In contrast, the expression of HLA-ABC and HLA-DR was completely abrogated, with less than 0.5% positive cells even after cytokine stimulation.

Furthermore, we used the corresponding isotypes and calculated the relative fluorescence intensity (RFI) by subtracting the isotype fluorescence to eliminate signals representing nonspecific binding. As shown in FIG. 2c, we observed that HLA-ABC and HLA-DR antigens were completely depleted from the CiGEnCΔHLA cell surface compared to the unmodified CiGEnC cell surface. Moreover, confocal analysis showed the ablation of not only HLA-ABC and HLA-DR but also HLA-DP and HLA-DQ in CiGEnCΔHLA cells after cytokine stimulation, suggesting complete depletion of all class I and II HLA antigens (FIG. 2d).

To confirm this total ablation of HLA antigens, we checked the expression of HLA-A2 on unmodified CiGEnC and CiGEnCΔHLA cells (FIG. 2e). Then, we used chimeric antigen receptor (CAR) technology to redirect the antigen specificity of CD8+ T cells toward the HLA-A2 antigen (FIG. 2f). We cocultured HLA-A2-specific CAR-T cells with CiGEnC or CiGEnCΔHLA cells and observed high cytotoxicity of the CD8+ CAR-T cells toward unmodified CiGEnC but not CiGEnCΔHLA cells (FIG. 2g).

### Example 3: CRISPR/Cas9 Editing does not Impair the Endothelial Phenotype in CiGEnCΔHLA Cells

CiGEnC have been previously characterized and are very similar to microvascular glomerular endothelial cells in terms of phenotype after one week of differentiation at 37° C. (Satchell, S. C. et al. Kidney Int. 69, 1633-1640 (2006).)

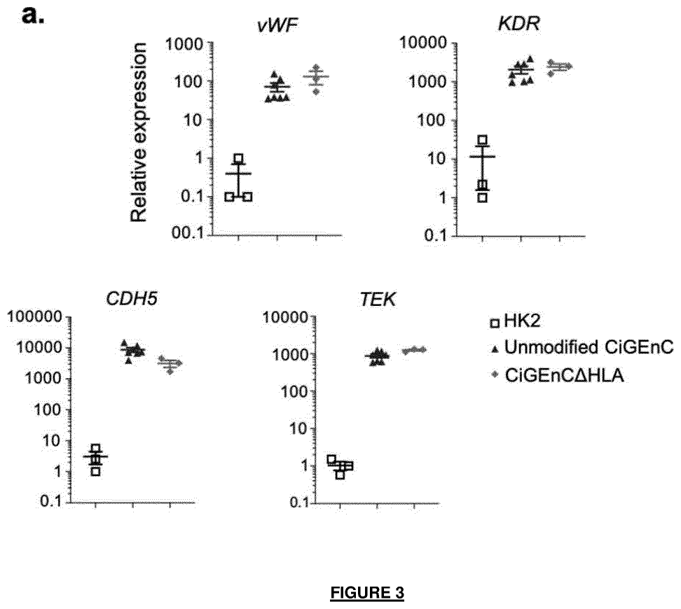

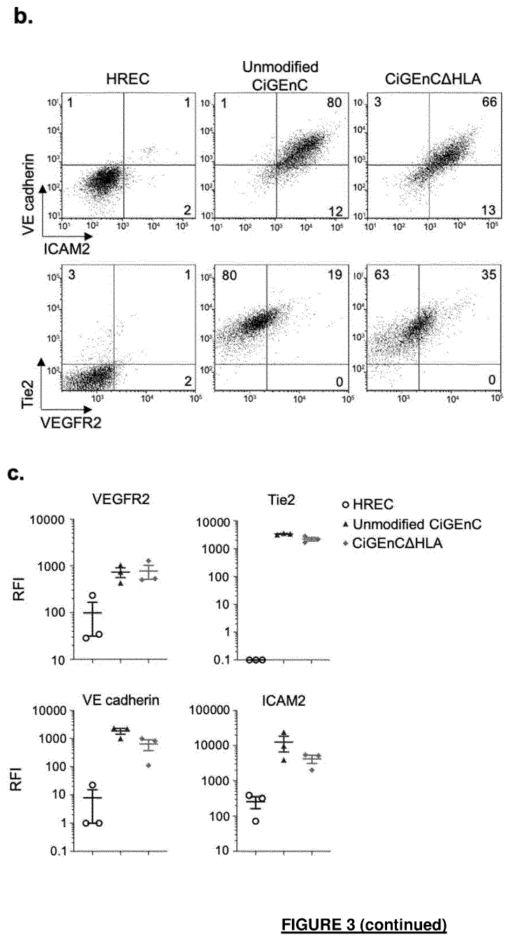

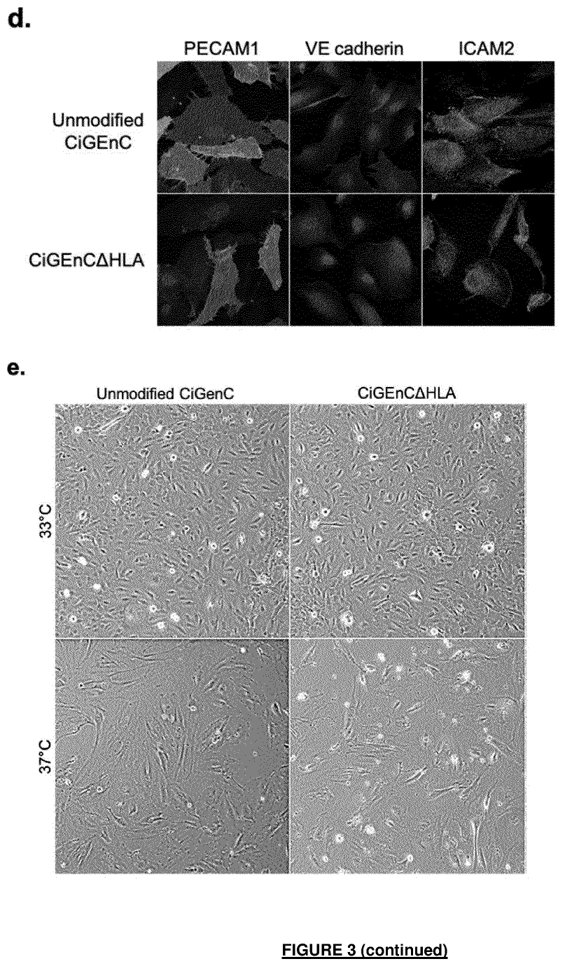

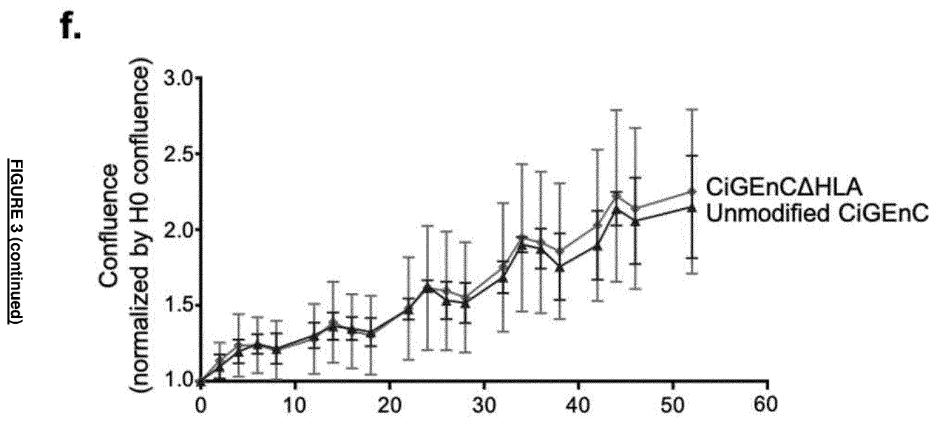

At the mRNA level, compared to human renal epithelial cells (HRECs), CiGEnCΔHLA and parental CiGEnC cells showed high expression levels of glomerular endothelial genes such as vWF, KDR, CDH5 and TEK (FIG. 3a).

Thus, we determined whether the CiGEnCΔHLA clone was still able to produce specific glomerular endothelial markers, such as VE cadherin, ICAM2, Tie2 and VEGFR2, using FACS analysis. As shown in FIGS. 3b and 3c, both unmodified CiGEnC cells and CiGEnCΔHLA cells similar levels of VE cadherin, ICAM2, Tie2 and VEGFR2 expression compared to HRECs. Immunofluorescence analyses confirmed the expression of PECAM1, VE cadherin and ICAM2 on the CiGEnCΔHLA cell surface (FIG. 3d). At both 33ºC and 37° C., CiGEnC retained features of early-passage primary glomerular endothelial cells in culture, including small size, homogeneity, and formation of “cobblestone” monolayers.

To test whether CRISPR/Cas9 gene editing would affect cell morphology, we first examined cells by phase-contrast microscopy. As expected, CiGEnCΔHLA cells were viable in culture and retained the morphologic features of unmodified CiGEnC (FIG. 3e). Thus, serially passaged CiGEnCΔHLA cells maintained the same proliferative profile as unmodified CiGEnC at 33° C. with loss of proliferation at 37° C., as expected (FIG. 3f).

### Example 4: Non-HLA Antibody Detection Assay (NHADIA)

### Patient Baseline Characteristics

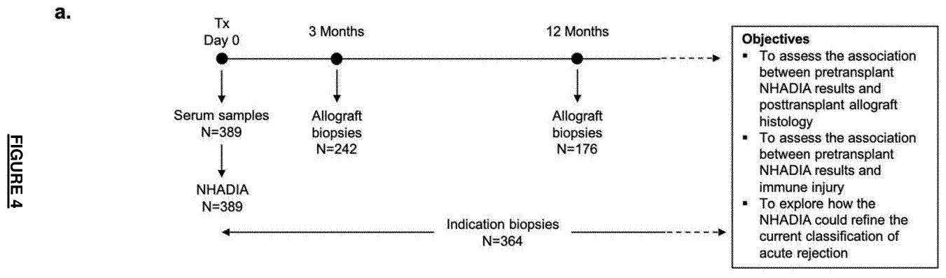

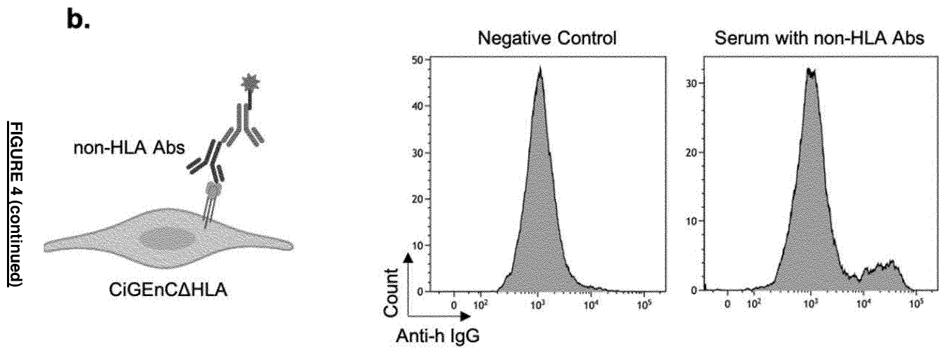

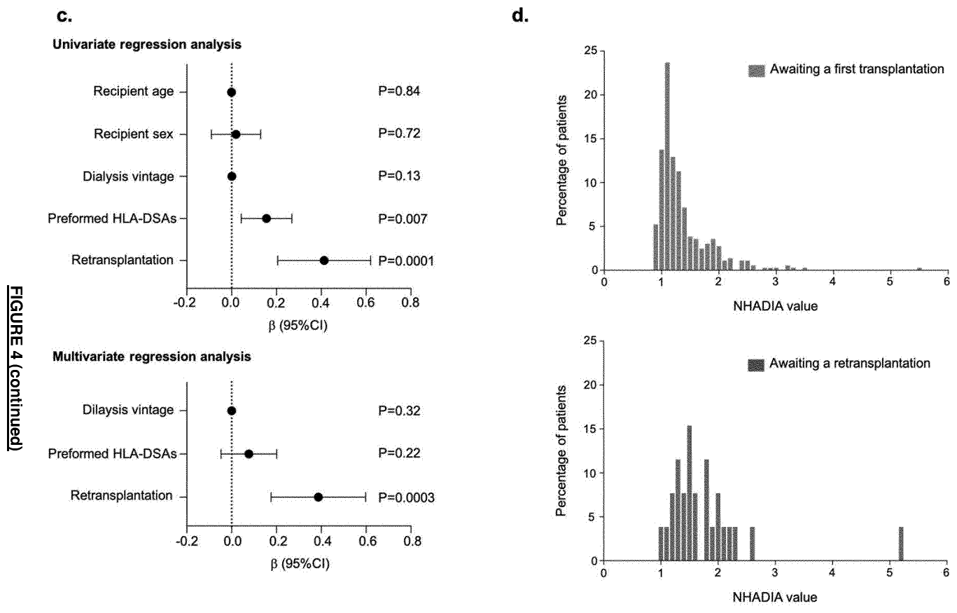

According to our inclusion criteria, 389 serum samples collected immediately before transplantation (Day 0 (D0)) from 389 unselected patients consecutively transplanted between January 2012 and June 2017 were available (FIG. 4a). Patient and donor characteristics at the time of transplantation are summarized in Table 1. The mean recipient age was 53.7±14.6 years, 64% were male, 21.6% received a kidney from a living donor, 6.7% had a previous kidney transplantation, and 68% of recipients (17.6%) had preformed HLA-DSAs.

TABLE 1
Patient and transplant characteristic
Table 1: Patient and transplant chracteristics

| | All | | NHADIA ≤1.87 | | NHADIA >1.87 | | |
|---|---|---|---|---|---|---|---|
| Population | N = 389 | | N = 332 | | N = 57 | | P value |
| Recipient characteristics | | | | | | | |
| Men, n (%) | 249 | (64) | 213 | (64.2) | 36 | (63.2) | 0.7495 |
| Age at transplantation (yr), mean ± sd | 53.7 ± 14.6 | | 54.0 ± 14.5 | | 52.0 ± 15.0 | | 0.8823 |
| Cause of ESKD | | | | | | | 0.9436 |
| Glomerulonephritis, n (%) | 72 | (18.5) | 60 | (18.1) | 12 | (21.1) | |
| Diabetes, n (%) | 42 | (10.8) | 35 | (10.5) | 7 | (12.3) | |
| Cystic/hereditary/congenital, n (%) | 114 | (29.3) | 97 | (29.2) | 17 | (29.8) | |
| Hypertension, n (%) | 41 | (10.5) | 36 | (10.8) | 5 | (8.8) | |
| Interstitial nephritis, n (%) | 33 | (8.5) | 29 | (8.7) | 4 | (7.0) | |
| Miscellaneous conditions, n (%) | 14 | (3.6) | 11 | (3.3) | 3 | (5.3) | |
| Etiology uncertain, n (%) | 73 | (18.8) | 64 | (19.3) | 9 | (15.8) | |
| Previous kidney transplantation, n (%) | 26 | (6.7) | 17 | (5.1) | 9 | (15.8) | 0.0069 |
| Time since dialysis onset (mth), mean ± sd | 39.7 ± 42 | | 39.0 ± 40.7 | | 44.4 ± 48.9 | | 0.4961 |
| Preemptive kidney transplantation, n (%) | 66 | (17.0) | 56 | (16.9) | 10 | (17.5) | 0.8505 |
| Transplant variables | | | | | | | |
| Donor age (yr), mean ± sd | 58.1 ± 17.1 | | 58.3 ± 17.0 | | 56.9 ± 17.9 | | 0.5993 |
| Living donors, n (%) | 84 | (21.6) | 72 | (21.7) | 12 | (21.1) | 1.000 |
| Deceased donors (DDs), n (%) | 305 | (78.4) | 260 | (78.3) | 45 | (78.9) | 1.000 |
| SCD, n (%) | 123 | (31.6) | 104 | (40.0) | 19 | (42.2) | 0.8695 |
| ECD, n (%) | 182 | (46.8) | 156 | (60.0) | 26 | (57.8) | 0.8695 |
| Cold ischemia time for DDs (h), mean ± sd | 20.1 ± 7.4 | | 19.9 ± 7.4 | | 20.7 ± 7.7 | | 0.5598 |
| Bitransplantation, n (%) | 47 | (12.1) | 43 | (13.0) | 4 | (7) | 0.2722 |
| Delayed graft function, n (%)^{a} | 71 | (18.6) | 63 | (19.4) | 8 | (14.0) | 0.4599 |
| Immunology | | | | | | | |
| Preformed DSAs, MFI >500, n (%) | 120 | (30.8) | 95 | (28.6) | 25 | (43.9) | 0.0291 |
| Preformed DSAs, MFI >1000, n (%) | 79 | (20.3) | 65 | (19.6) | 14 | (24.6) | 0.3774 |
| HLA-A, HLA-B and HLA-DR incompatibilities, | 3.3 ± 1.5 | | 3.4 ± 1.5 | | 3.2 ± 1.4 | | 0.3854 |
| mean ± sd | | | | | | | |
| Induction therapy | | | | | | | |
| Thymoglobuline, n (%) | 149 | (38.3) | 118 | (35.5) | 31 | (54.4) | 0.0080 |
| Anti-CD25 antibody, n (%) | 227 | (58.4) | 204 | (61.4) | 23 | (40.4) | 0.0035 |
| None, n (%) | 13 | (3.3) | 10 | (3.0) | 3 | (5.3) | 0.4167 |
| Follow-up | | | | | | | |
| Length of follow-up (yr), median (IQR) | 55.5 | (38.8-62.8) | 53.3 | (38.4-62.8) | 57.2 | (48.3-62.9) | 0.2981 |

| | | | | |
|---|---|---|---|---|
| 3 months | N = 377 | N= | N= | |
| Creatininemia (μmol/L), mean ± sd | 141.5 ± 60.4 | 140.3 ± 61.6 | 148.6 ± 53.5 | 0.1013 |
| eGFR (mL/min/1.73 m²), mean ± sd | 51.1 ± 20.1 | 51.6 ± 20.1 | 48.0 ± 20.1 | 0.1533 |
| 12 months | N = 365 | N= | N= | |
| Creatininemia (μmol/L), mean ± sd | 139.9 ± 50.2 | 137.6 ± 49.1 | 152.5 ± 54.9 | 0.0268 |
| eGFR (mL/min/1.73 m²), mean ± sd | 50.6 ± 19.8 | 51.3 ± 19.8 | 46.4 ± 19.9 | 0.0358 |
| 60 months | N = 185 | | | |
| Creatininemia (μmol/L), mean ± sd | 140.6 ± 56.2 | 135.9 ± 54.7 | 163.1 ± 58.9 | 0.0055 |
| eGFR (mL/min/1.73 m²), mean ± sd | 50.8 ± 20.7 | 52.1 ± 20.3 | 44.4 ± 21.7 | 0.0370 |

Abbreviations: n, number; yr, years; sd, standard deviation; ESKD, end-stage kidney disease; mth, months; SCD, standard criteria donors; ECD, expanded criteria donors; DDs, deceased donors; h, hours; DSAs, donor-specific antibodies; IQR, interquartile range; eGFR, estimated glomerular filtration rate with the MDRD formula.
^{a}Data unavailable for 7 patients

Pretransplant NHADIA Results are Associated with Retransplantation Status

The cell-based NHADIA was performed using the 389 pretransplant serum samples with CiGEnCΔHLA cells as the targets, as described in the Methods section. FIG. 4b shows a representative result compared to that of a negative control.

The median (interquartile range (IQR)) value of the NHADIA was 1.26 (1.14-1.56). Linear regression analysis was performed to identify pretransplant determinants of the NHADIA value (FIG. 4c) and identified previous transplantation as the main determinant of the NHADIA result. Indeed, the median NHADIA (IQR) value was 1.24 (1.13-1.52) among the 363 patients awaiting their first transplantation (FIG. 4d, top panel) and 1.62 (1.41-2.03) among the 26 patients awaiting a retransplantation (P<0.0001) (FIG. 4d, lower panel).

Pretransplant NHADIA Results are Associated with Microvascular Inflammation (MVI)

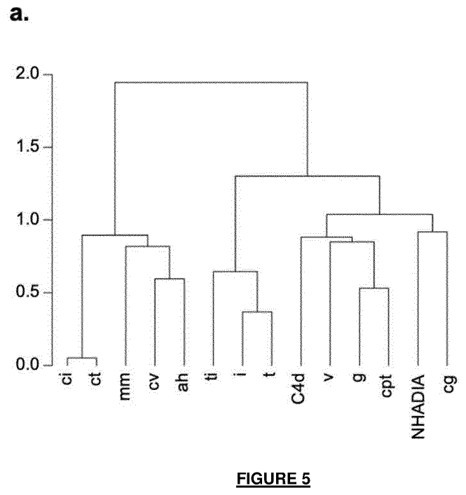

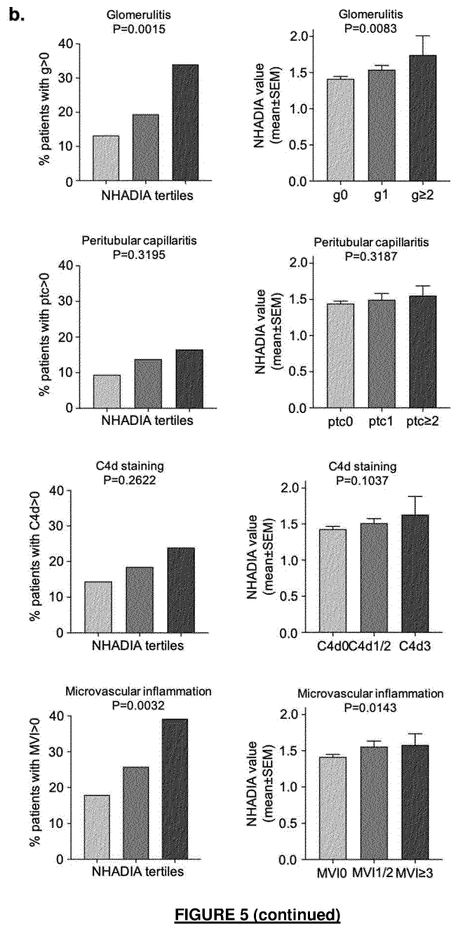

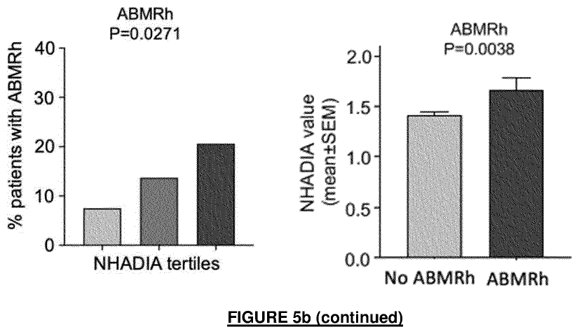

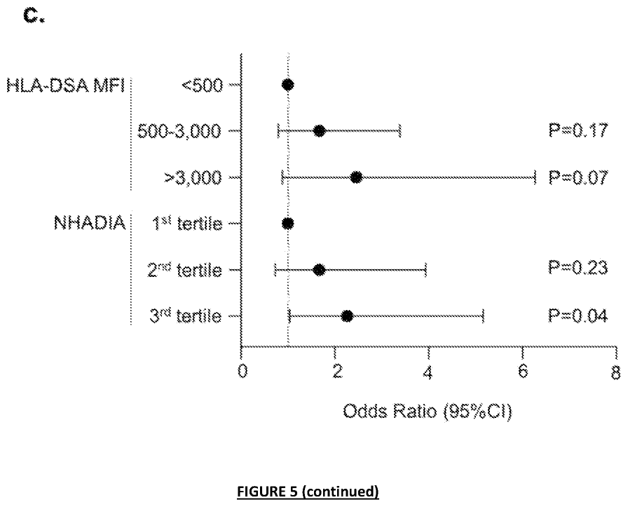

A total of 951 adequate kidney allograft biopsies were performed during follow-up, including 510 biopsies performed at 3 months (N=298) or 12 months (N=212) post-transplantation and 441 additional indication biopsies performed at a median time of 7.1 (1.3-14.7) months after transplantation. The biopsy characteristics are summarized in Table 2. We first addressed the association of NHADIA values with allograft histology at 3 months post-transplantation. Unsupervised clustering analysis revealed that the NHADIA results preferentially clustered with histological features of ABMR (FIG. 5a). Stratification of biopsies according to NHADIA tertiles (FIG. 5b, left) demonstrated that higher levels of non-HLA Abs were positively correlated with an increased presence of glomerulitis (g, P=0.0015), MVI (P=0.0032) and ABMRh (P=0.027). In addition, increasing severity of glomerulitis (P=0.0083) and MVI (P=0.0143) were associated with increased NHADIA values (FIG. 5b, right). Furthermore, biopsies showing ABMRh at 3 months were associated with elevated NHADIA results (P=0.0038). Finally, multivariate regression analysis demonstrated that increased NHADIA values were associated with ABMRh at 3 months independent of HLA-DSAs (FIG. 5c). Similar patterns were observed at 12 months post-transplantation. Of note, other acute Banff lesion interstitial infiltrate (i), tubulitis (t), chronic vascular changes (cv), arteriolar hyalinosis (ah), interstitial fibrosis (ci), and tubular atrophy (ct) scores were similar among the 3 groups (data not shown).

TABLE 2
Description of kidney biopsies performed at 3 months, 12 months or any other time lapse after transplatantation

| | All Biopsies | | M 3 | | M 12 | | Other time | |
|---|---|---|---|---|---|---|---|---|
| Variables | N = 951 | | N = 298 | | N = 212 | | N = 441 | |
| Biopsy indications (NA = 5) | | | | | | | | |
| Screening biopsy, n (%) | 583 | (64) | 264 | (88.9) | 202 | (95.3) | 117 | (26.8) |
| Acute kidney injury, n (%) | 278 | (29.4) | 30 | (10.1) | 6 | (2.8) | 242 | (55.4) |
| BK viremia, n (%) | 23 | (2.4) | 3 | (1.0) | 0 | (0) | 20 | (4.6) |
| De novo DSAs, n (%) | 6 | (0.6) | 0 | (0) | 0 | (0) | 6 | (1.4) |
| Control post acute rejection, n (%) | 52 | (5.5) | 0 | (0) | 4 | (1.9) | 48 | (11.0) |
| Other, n (%) | 4 | (0.4) | 0 | (0) | 0 | (0) | 4 | (0.9) |
| Time from transplant to biopsy (mth), median (IQR) | 5.0 | [3-12.4] | 3.1 | [2.9-3.2] | 12.1 | [11.8-12.4] | 7 | [1.3-14.7] |
| DSAs at the time of biopsy, n (%) | 125 | (13.1) | 32 | (10.7) | 22 | (10.4) | 71 | (16.1) |
| Pathological lesions | | | | | | | | |
| Glomerulitis (NA = 12) | | | | | | | | |
| Score > 0, n (%) | 255 | (27.2) | 65 | (22.0) | 52 | (24.9) | 138 | (31.8) |
| Score, mean ± sd | 0.4 ± 0.7 | | 0.3 ± 0.6 | | 0.3 ± 0.6 | | 0.5 ± 0.8 | |
| Peritubular capillaritis (NA = 8) | | | | | | | | |
| Score > 0, n (%) | 165 | (17.5) | 39 | (13.1) | 18 | (8.6) | 108 | (24.7) |
| Score, mean ± sd | 0.3 ± 0.7 | | 0.2 ± 0.6 | | 0.1 ± 0.5 | | 0.5 ± 0.9 | |
| MVI (NA = 13) | | | | | | | | |
| Score > 0, n (%) | 303 | (32.3) | 81 | (27.4) | 57 | (27.3) | 165 | (38.1) |
| Score, mean ± sd | 0.7 ± 1.3 | | 0.5 ± 1.0 | | 0.5 ± 1.0 | | 0.9 ± 1.5 | |
| Tubulitis (NA = 13) | | | | | | | | |
| Score > 0, n (%) | 173 | (18.4) | 36 | (12.3) | 26 | (12.5) | 111 | (25.3) |
| Score, mean ± sd | 0.4 ± 0.9 | | 0.2 ± 0.7 | | 0.2 ± 0.7 | | 0.5 ± 1.0 | |
| Interstitial infiltrate (NA = 14) | | | | | | | | |
| Score > 0, n (%) | 68 | (7.3) | 19 | (6.5) | 4 | (1.9) | 45 | (10.3) |
| Score, mean ± sd | 0.1 ± 0.5 | | 0.1 ± 0.3 | | 0.0 ± 0.3 | | 0.2 ± 0.6 | |
| Vasculitis (NA = 100) | | | | | | | | |
| Score > 0, n (%) | 33 | (3.9) | 8 | (2.9) | 0 | (0) | 26 | (6.4) |
| Score, mean ± sd | 0.1 ± 0.4 | | 0.0 ± 0.3 | | 0.0 ± 0.0 | | 0.1 ± 0.5 | |
| C4d (NA = 28) | | | | | | | | |
| Score > 0, n (%) | 214 | (23.2) | 54 | (18.8) | 41 | (20.1) | 119 | (27.6) |
| Score, mean ± sd | 0.4 ± 0.8 | | 0.3 ± 0.6 | | 0.3 ± 0.7 | | 0.5 ± 0.9 | |
| Chronic vascular changes (NA = 115) | | | | | | | | |
| Score > 0, n (%) | 639 | (76.4) | 186 | (69.9) | 137 | (72.9) | 316 | (82.7) |
| Score, mean ± sd | 1.5 ± 1.1 | | 1.3 ± 1.1 | | 1.4 ± 1.1 | | 1.6 ± 1.0 | |
| Arteriolar hyalinosis (NA = 22) | | | | | | | | |
| Score > 0, n (%) | 699 | (75.2) | 206 | (71.3) | 155 | (74.9) | 338 | (78.1) |
| Score, mean ± sd | 1.2 ± 1.0 | | 1.1 ± 0.9 | | 1.2 ± 0.9 | | 1.4 ± 1.0 | |
| Allograft glomerulopathy (NA = 16) | | | | | | | | |
| Score > 0, n (%) | 30 | (3.2) | 5 | (1.7) | 7 | (3.4) | 18 | (4.1) |
| Score, mean ± sd | 0.0 ± 0.3 | | 0.0 ± 0.1 | | 0.0 ± 0.2 | | 0.1 ± 0.4 | |
| IFTA (NA = 113) | | | | | | | | |
| Score > 0, n (%) | 544 | (64.9) | 132 | (53) | 123 | (64.4) | 289 | (72.6) |
| Score, mean ± sd | 1.1 ± 1.0 | | 0.8 ± 0.9 | | 1.0 ± 0.9 | | 1.2 ± 1.0 | |
| Acute rejections^{a} | | | | | | | | |
| ABMR, n (%) (NA = 3) | 80 | (8.4) | 17 | (5.7) | 10 | (4.7) | 53 | (12.0) |
| ABMRh, n (%) (NA = 0) | 183 | (19.2) | 41 | (13.7) | 30 | (14.2) | 112 | (25.4) |
| TCMR, n (%) (NA = 3) | 29 | (3.1) | 3 | (1) | 0 | (0) | 26 | (5.9) |

Abbreviations: NA, data unavailable; DSAs, donor-specific antibodies; MVI, microvascular inflammation; IFTA, interstitial fibrosis and tubular atrophy; ABMR, acute antibody-mediated rejection; TCMR, T cell-mediated rejection.
^{a}According to Banff classification 2019 of kidney transplant biopsies.

### Pretransplant NHADIA Results Predict ABMRh

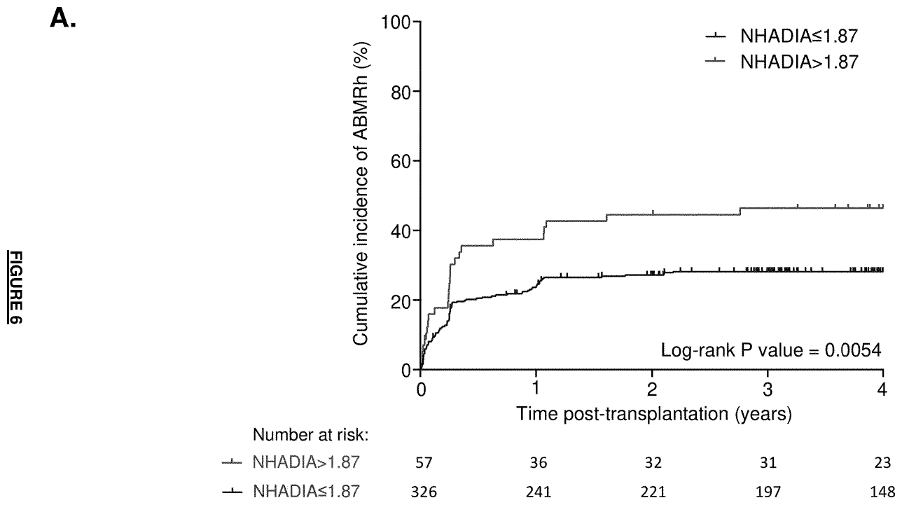

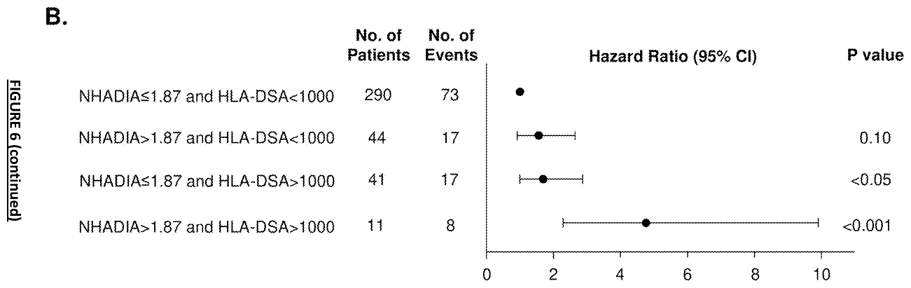

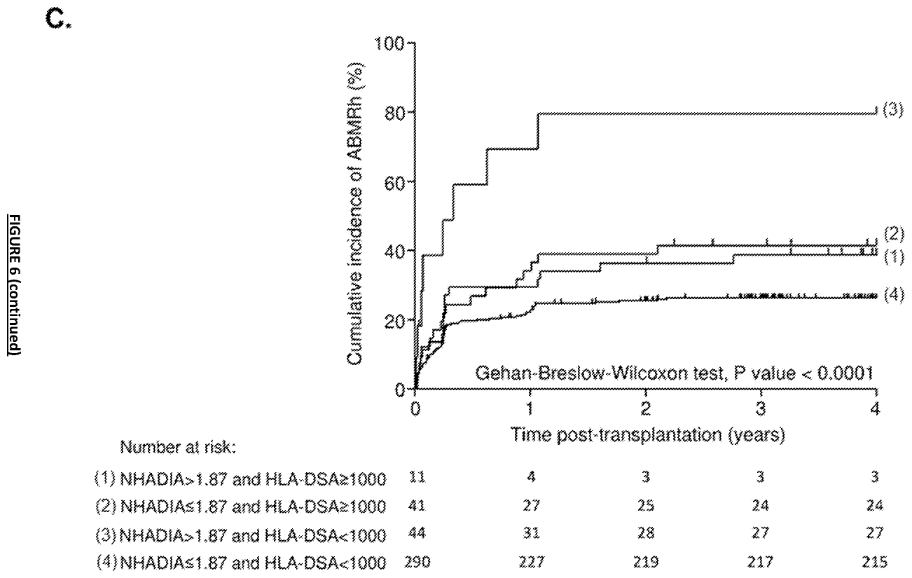

We next sought to determine the best pretransplant NHADIA threshold for predicting posttransplant ABMRh. We plotted log-rank test P-values of the comparison of time to ABMRh occurrence for various NHADIA thresholds, and we observed an inverted peak corresponding to a NHADIA value of 1.87 (FIG. 6d). Thus, we defined this 1.87 NHADIA threshold as the indicator for non-HLA Ab presence. FIG. 6a illustrates that the NHADIA threshold of 1.87 strongly discriminated between patients who were at risk of ABMRh and those who were not (P=0.0055, log-rank test).

To investigate whether non-HLA Abs assessed by the NHADIA can act in synergy with HLA-DSAs, resulting in an increased risk of ABMRh, patients were subsequently stratified into four groups according to their statuses for HLA-DSAs and non-HLA Abs. Multivariate Cox regression analysis demonstrated that compared to patients with no HLA-DSAs or non-HLA Abs, patients with non-HLA Abs but no HLA-DSAs had a non-significantly increased risk of ABMRh (HR 1.56, 95% CI 0.92-2.65, P=0.10), patients with HLA-DSAs but no non-HLA Abs had a significantly increased risk of ABMRh (HR 1.70, 95 Cl 1.00-2.88, P<0.05), and patients with both HLA-DSAs and non-HLA Abs at the time of transplantation had the highest risk of ABMRh (HR 4.77, 95 CI 2.29-9.91, P<0.001) (FIG. 6b). FIG. 6c depicts the cumulative incidence of ABMRh for the four groups. Patients with neither type of Abs experienced the best outcome, with a cumulative ABMRh incidence of 26.3% at 4 years. In contrast, patients with HLA-DSAs and non-HLA Abs experienced the poorest outcome, with a cumulative ABMRh incidence of 79.5% at 4 years. Patients with HLA-DSAs but no non-HLA Abs and patients with non-HLA Abs but no HLA-DSAs displayed intermediate risk with cumulative ABMRh incidences of 41.5% and 38.7% at 4 years, respectively (FIG. 6c).

Reclassification of Patients with Non-HLA Abs According to Banff Classification Updates

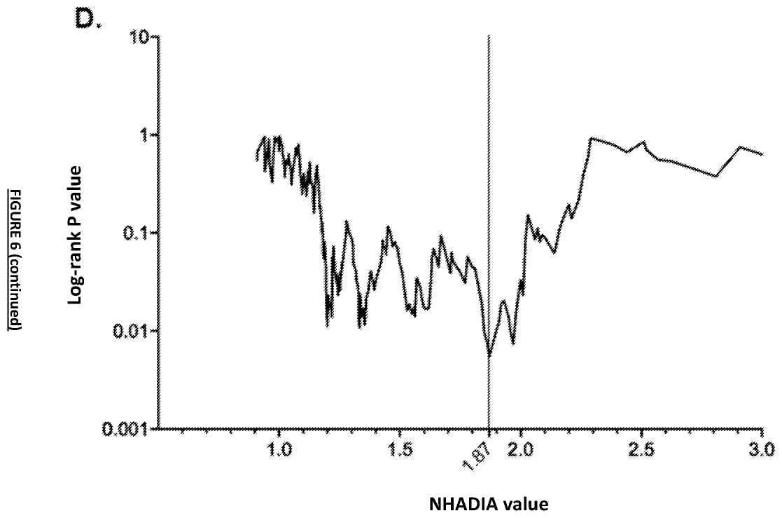

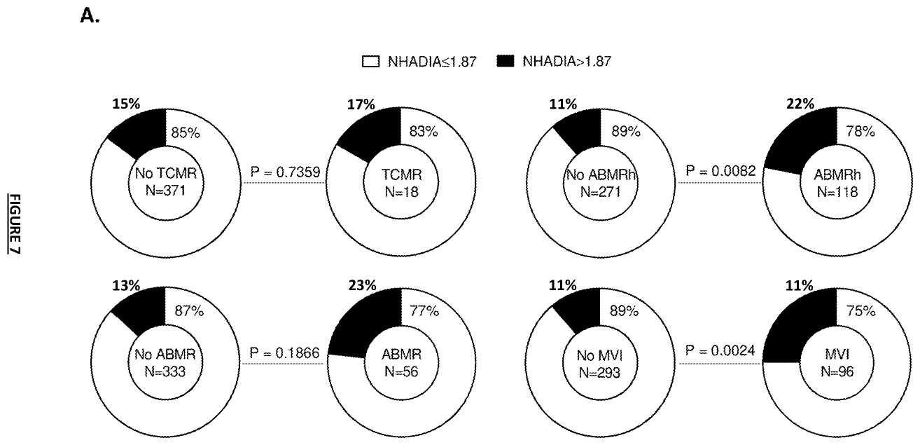

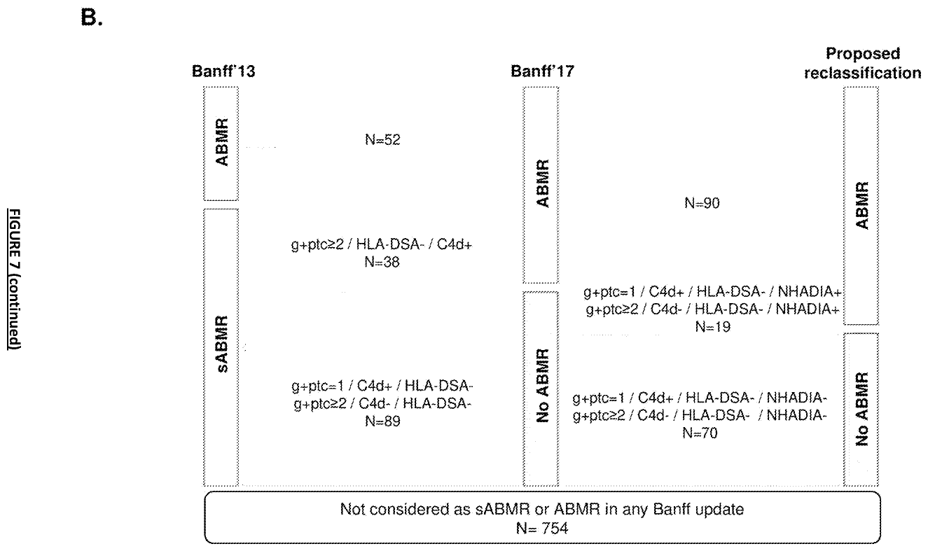

According to the Banff 2017 classification, acute rejection developed in 74 patients: 18 patients had T cell-mediated rejection, and 56 had antibody-mediated rejection. No evident association with NHADIA status was observed in these 2 entities (FIG. 7a). However, the proportion of patients with a NHADIA result>1.87 was significantly greater among those with ABMRh (P=0.0082) and MVI (P=0.0024), two conditions that are not sufficient to diagnose ABMR according to the Banff 2017 classification, due to the absence of demonstration of pathogenic antibodies (FIG. 7a).

To assess whether NHADIA status can be used to evolve definitions of ABMR, we performed a landmark analysis of all our biopsies. Among the 933 included biopsies, 179 biopsies were classified as ABMR or suspicious for ABMR (sABMR) by at least one of the 2013 or 2017 updates of the Banff classification (FIG. 7b). A total of 127 biopsies were classified as sABMR by Banff′13 because of morphologic and serologic evidence (v>0 and HLA-DSA positivity) or immunohistologic evidence (v>0 and C4d positivity, or at least moderate MVI). Thus, mainly due to the use of at least moderate MVI as the second criterion, Banff′13 contained the largest number of biopsies diagnosed with SABMR or ABMR (n=127 and n=52, respectively). As the suspicious category was eliminated in Banff′17, 89/127 (70.1%) Banff′13 sABMR biopsies were reclassified as no ABMR by Banff′17 due to the absence of the second criterion (C4d positivity with positive g or peritubular capillaritis (cpt)) or the third criterion (C4d negative, HLA-DSA negative with MVI). The other 38/127 (29.9%) Banff′13 sABMR biopsies were reclassified as ABMR by Banff′17 because of positive C4d staining and MVI in the absence of HLA-DSAs. Of the 52 Banff′13 ABMR biopsies, all remained classified as ABMR by Banff′17. The NHADIA, by detecting the presence of non-HLA Abs directed against the glomerular endothelium, may be an alternative way to fulfill the 3rd criterion of the Banff classification. We thus proposed reclassifying patients with a NHADIA result>1.87 but no HLA-DSAs as ABMR in an evolution of the Banff classification. By doing so, 19 biopsies classified as no ABMR by Banff′17 would be considered ABMR biopsies due to the presence of non-HLA Abs.

### Example 5: Discussion

Antibody-mediated rejection (ABMR) is thought to be the main determinant of kidney allograft failure in the long term (Loupy, A. & Lefaucheur, C New England Journal of Medicine (2018). doi:10.1056/NEJMra1802677). However, its accurate diagnosis remains challenging, as exemplified by the regular iterations of its diagnostic criteria endorsed by the international Banff classification (Haas, M. et al. American Journal of Transplantation (2018). doi:10.1111/ajt.14625; Loupy, A. et al. American Journal of Transplantation (Am J Transplant, 2020). doi:10.1111/ajt. 15898; Haas, M. et al. Am. J. Transplant. 14, 272-283 (2014).)

The main diagnostic challenge lies in the lack of demonstration of pathogenic antibodies in the serum of many patients with ABMRh. HLA-DSAs have long been considered to be the main source of pathogenic antibodies, but recent large cohort assessments demonstrated that 40-60% of ABMRh cases are HLA-DSA negative at the time of biopsy, suggesting the involvement of alternative mechanisms of allograft injury (Senev, A. et al. Am. J. Transplant. 19, 763-780 (2019); Koenig, A. et al. Nat. Commun. 10, 5350 (2019); Luque, S. et al. Am. J. Transplant. 19, 368-380 (2019).)

In this study, we developed an innovative method that allowed the identification of non-HLA Abs that are not only associated with ABMRh independent of HLA-DSAs but also seem to act synergistically with HLA-DSAs to induce ABMRh.

CiGEnC was previously as targets for detecting non-HLA Abs associated with ABMRh (Delville, M. et al. J. Am. Soc. Nephrol. 30, 692-709 (2019).) However, the basal expression of HLA antigens on their surface limited their application for non-HLA Abs detection in patients without circulating anti-HLA Abs. To tackle this obstacle, a CRISPR/Cas9 strategy was sequentially applied to delete both the B2M and CIITA genes by a nonhomologous end-joining pathway to obtain a CiGEnCΔHLA clone. CiGEnCΔHLA cells remained undistinguishable from the parental cell line in terms of morphology and phenotype and allowed us to develop a new assay (NHADIA).

The evaluation of the NHADIA in an unselected cohort of kidney transplant recipients (KTRs) revealed that non-HLA Abs were increased in patients who underwent previous kidney transplantation, supporting the role of allosensitization to minor histocompatibility antigens, a sensitization that has been shown to affect long-term graft outcomes (Reindl-Schwaighofer, R. et al. Lancet (London, England) 393, 910-917 (2019); Steers, N. J. et al. N. Engl. J. Med. 380, 1918-1928 (2019).)

A large array of non-HLA antigens, including polymorphic antigens that differ between the recipient and donor, such as the recently described genomic mismatch at the LIMS1 locus (Steers, N. J. et al. N. Engl. J. Med. 380, 1918-1928 (2019) have already been suspected as targets for non-HLA sensitization, and other candidates have been described in recent decades, including anti-AT1R Abs (Dragun, D. et al. N. Engl. J. Med. 352, 558-569 (2005); Lefaucheur, C. et al. Kidney Int. 96, 189-201 (2019).) anti-ETAR Abs, polyreactive natural Abs(See, S. B. et al. J. Am. Soc. Nephrol. 29, 1761 LP-1770 (2018)) and many others (Delville, M. et al. J. Am. Soc. Nephrol. 30, 692-709 (2019).; Anglicheau, D., Delville, M. & Lamarthee, B. Nephrol. Ther. (2019). doi:10.1016/j.nephro.2019.03.003; Delville, M., Charreau, B., Rabant, M., Legendre, C. & Anglicheau, D. Human Immunology 77, 1055-1062 (2016); Jackson, A. M., Delville, M., Lamarthée, B. & Anglicheau, D. Hum. Immunol. (2019). doi:10.1016/J.HUMIMM.2019.04.014) More generally, the overall non-HLA mismatch burden between a donor and recipient was recently associated with poor graft survival in a genome-wide analysis (Reindl-Schwaighofer, R. et al. Lancet (London, England) 393, 910-917 (2019).). Interrogating the whole genome of donors and recipients, Reindl-Schwaighofer et al revealed the large number of mismatches that might induce a humoral alloimmune response in the recipient. Indeed, they identified a median value of 1,892 nonsynonymous single-nucleotide polymorphism (snSNP) mismatches in immune-accessible proteins between donors and recipients. Interestingly, the degree of nsSNP mismatch was independently associated with graft loss, thus suggesting that this tremendous diversity should be taken into account when addressing the extent of immunological injury to the graft. In this respect, we believe that our cell-based assay, which is not a candidate-driven approach but integrates the diversity of plausible targets, may improve the diagnosis of non-HLA Ab-mediated cell injury. It remains to be seen whether combination of the NHADIA with proteome-wide Ab screens such as Cellular Indexing of Transcriptomes and Epitopes by Sequencing (CITE-seq) (Stoeckius, M. et al. Nat. Methods 14, 865-868 (2017).), which assesses over one hundred surface proteins at the same time, can be used effectively to uncover new histocompatibility antigens expressed by the glomerular endothelium. These potential antigen screening approaches could pave the way for personalized medicine and, more specifically, for improved organ matching.

It was observed that the pretransplant NHADIA value correlated with MVI lesions on the kidney graft at 3 months and 12 months after transplantation and was associated with the risk of developing ABMRh. MVI has been associated with poor kidney graft outcomes in several studies, and its deleterious impact has been observed even in the absence of HLA-DSAs (Loupy, A. et BMJ 366, 14923 (2019); Callemeyn, J. et al. J. Am. Soc. Nephrol. 31, 2168-2183 (2020).). Interestingly, it was observed a biological gradient between the pretransplant value of the NHADIA and the occurrence and severity of MVI on kidney biopsies, suggesting a causal role for these non-HLA Abs as mediators of endothelial injury. A multivariate regression analysis confirmed that pretransplant non-HLA Abs and HLA-DSAs were associated with posttransplant MVI and that increasing levels of either type of Abs independently increased the risk of MVI. Of note, the NHADIA value was specifically associated with MVI but not associated with tubulitis or interstitial inflammation, two features of T cell-mediated rejection, again supporting the instrumental role for non-HLA Abs in the development of MVI. The biological plausibility of the causal link is further supported by the strong association between high NHADIA values and glomerulitis, much stronger than that with peritubular capillaritis, confirming that non-HLA Abs recognize antigens specifically expressed by the glomerular endothelium. By using glomerular endothelial cells, a test was created with high specificity for detecting Abs that target antigens specifically expressed on glomerular endothelial cells. These observations are in line with the recent observation obtained by single-cell RNA sequencing that even among kidney endothelial cells, glomerular endothelial cells express distinct gene sets compared to vasa-recta endothelial cells and peritubular endothelial cells (Lake, B. B. et al. Nat. Commun. 10, 2832 (2019).)

The NHADIA may have consequences for firmly diagnosing a number of unresolved suspected cases of ABMR. MVI on kidney allograft biopsies is a key feature of ABMR that has remained a cornerstone parameter in the consecutive Banff classifications. Nevertheless, the 3^{rd} criterion of the Banff classification, based on serological evidence of DSAs or indirect proof provided by C4d-positive staining or validated transcripts, remains a prerequisite to establish a definite diagnosis of ABMR (Loupy, A. et al. American Journal of Transplantation (Am J Transplant, 2020). doi:10.1111/ajt.15898). A considerable number of kidney biopsies display some of the pathological features of ABMR (ABMRh), without satisfying the 3^{rd} criterion, theoretically preventing a physician from diagnosing ABMR. In a recent study, Calleymen et al showed that the previous entity of “suspected ABMR” described by the 2013 Banff classification, now considered “no ABMR”, is mainly represented by ABMRh lacking C4d staining and HLA-DSA positivity. However, this entity was significantly associated with impaired allograft survival in their cohort. This observation leads to the conclusion that ABMRh without HLA-DSAs and/or C4d staining should not be neglected in clinical practice (Callemeyn, J. et al. Am. J. Transplant. Off. J. Am. Soc. Transplant. Am. Soc. Transpl. Surg. (2020). doi:10.1111/ajt.16474

Furthermore, Shinstock et al recently showed that almost 50% of cases with histologic features of ABMR lacking HLA-DSA positivity and C4d staining were considered ABMR by clinicians and were mainly treated as such, even though the Banff-based diagnoses would have concluded an absence of ABMR (Schinstock, C. A. et al. Am. J. Transplant. 19, 123-131 (2019).

This makes sense, as the 3rd criterion remains a challenging point in clinical practice. In the absence of HLA-DSAs, non-HLA Abs are suspected, but in the absence of a robust test, they often remain unrecognized. To address this issue, the Banff classification allows indirect proof of DSAs from C4d staining, which has poor sensitivity with up to 50% negative results in HLA-DSA-associated ABMR (Loupy, A. & Lefaucheur, C. New England Journal of Medicine (2018). doi:10.1056/NEJMra1802677), and validated transcripts in a kidney biopsy, which is not done in routine practice. Hence, a portion of authentic ABMR cases is currently misdiagnosed and consequently mistreated. The NHADIA, by detecting the presence and measuring the quantity of non-HLA Abs directed against the glomerular endothelium, may be viewed as an alternative way to fulfill the 3^{rd} criterion of the Banff classification, confirm involvement of antibodies and adjust treatment regimens to target antibodies.

In conclusion, in addition to providing new insights into graft injury mechanisms, the NHADIA has the potential to refine risk assessment prior to transplantation, demonstrate the involvement of antibodies at the time of alloimmune injury, improve acute rejection diagnosis and adjust therapeutic interventions by targeting detrimental antibodies.

### REFERENCES

Anglicheau, D., Delville, M. & Lamarthee, B. Non anti-HLA antibodies and acute rejection: A critical viewpoint. Nephrol. Ther. (2019). doi:10.1016/j.nephro.2019.03.003
Anglicheau, D. et al. Role of P-glycoprotein in cyclosporine cytotoxicity in the cyclosporine-sirolimus interaction. Kidney Int. 70, 1019-25 (2006).
Amrouche, L. et al. MicroRNA-146a in human and experimental ischemic AKI: CXCL8-dependent mechanism of action. J. Am. Soc. Nephrol. 28, 479-493 (2017).
Bestard, O. & Grinyó, J. Refinement of humoral rejection effector mechanisms to identify specific pathogenic histological lesions with different graft outcomes. American Journal of Transplantation 19, 952-953 (2019).
Callemeyn, J. et al. Transcriptional Changes in Kidney Allografts with Histology of Antibody-Mediated Rejection without Anti-HLA Donor-Specific Antibodies. J. Am. Soc. Nephrol. 31, 2168-2183 (2020).
Callemeyn, J. et al. Revisiting the changes in the Banff classification for antibody-mediated rejection after kidney transplantation. Am. J. Transplant. Off. J. Am. Soc. Transplant. Am. Soc. Transpl. Surg. (2020). doi:10.1111/ajt.16474
Christiaans, Maarten H. L.; Nieman, Fred; van Hooff, Johannes P.; van den Berg-Loonen, Ella M., Detection Of Hla Class I And II Antibodies By Elisa And Complement-Dependent Cytotoxicity Before And After Transplantation, Transplantation: Mar. 15, 2000—Volume 69—Issue 5—p 917-927
Delville, M., Charreau, B., Rabant, M., Legendre, C. & Anglicheau, D. Pathogenesis of non-HLA antibodies in solid organ transplantation: Where do we stand? Human Immunology 77, 1055-1062 (2016).
Delville, M. et al. Early acute microvascular kidney transplant rejection in the absence of anti-HLA antibodies is associated with preformed IgG antibodies against diverse glomerular endothelial cell antigens. J. Am. Soc. Nephrol. 30, 692-709 (2019).
Detrisac, C. J., Sens, M. A., Garvin, A. J., Spicer, S. S. & Sens, D. A. Tissue culture of human kidney epithelial cells of proximal tubule origin. Kidney Int. 25, 383-90 (1984).
Dragun, D. et al. Angiotensin II type 1-receptor activating antibodies in renal-allograft rejection. N. Engl. J. Med. 352, 558-569 (2005).
Haas, M. et al. The Banff 2017 Kidney Meeting Report: Revised diagnostic criteria for chronic active T cell-mediated rejection, antibody-mediated rejection, and prospects for integrative endpoints for next-generation clinical trials. in American Journal of Transplantation (2018). doi:10.1111/ajt.14625
Haas, M. et al. Banff 2013 Meeting Report: Inclusion of C4d-Negative Antibody-Mediated Rejection and Antibody-Associated Arterial Lesions. Am. J. Transplant. 14, 272-283 (2014).
Jackson, A. M., Delville, M., Lamarthee, B. & Anglicheau, D. Sensitization to endothelial cell antigens: Unraveling the cause or effect paradox. Hum. Immunol. (2019). doi:10.1016/J.HUMIMM.2019.04.014
Koenig, A. et al. Missing self triggers NK cell-mediated chronic vascular rejection of solid organ transplants. Nat. Commun. 10, 5350 (2019).
Lake, B. B. et al. A single-nucleus RNA-sequencing pipeline to decipher the molecular anatomy and pathophysiology of human kidneys. Nat. Commun. 10, 2832 (2019).
LeibundGut-Landmann, S. et al. Mini-review: Specificity and expression of CIITA, the master regulator of MHC class II genes. Eur. J. Immunol. 34, 1513-25 (2004).
Lefaucheur, C. et al. Non-HLA agonistic anti-angiotensin II type 1 receptor antibodies induce a distinctive phenotype of antibody-mediated rejection in kidney transplant recipients. Kidney Int. 96, 189-201 (2019).
Loupy, A. et al. The Banff 2019 Kidney Meeting Report (I): Updates on and clarification of criteria for T cell- and antibody-mediated rejection. in American Journal of Transplantation (Am J Transplant, 2020). doi:10.1111/ajt.15898
Loupy, A. & Lefaucheur, C. Antibody-mediated rejection of solid-organ allografts. New England Journal of Medicine (2018). doi:10.1056/NEJMra1802677
Loupy, A. et al. The Banff 2015 Kidney Meeting Report: Current Challenges in Rejection Classification and Prospects for Adopting Molecular Pathology. Am. J. Transplant. 17, 28-41 (2017).
Loupy, A. et al. Prediction system for risk of allograft loss in patients receiving kidney transplants: international derivation and validation study. BMJ 366, 14923 (2019).
Luque, S. et al. Value of monitoring circulating donor-reactive memory B cells to characterize antibody-mediated rejection after kidney transplantation. Am. J. Transplant. 19, 368-380 (2019).
Merola J, Reschke M, Pierce R W, Qin L, Spindler S, Baltazar T, Manes T D, Lopez-Giraldez F, Li G, Bracaglia L G, Xie C, Kirkiles-Smith N, Saltzman W M, Tietjen G T, Tellides G, and Pober J S, Progenitor-derived human endothelial cells evade alloimmunity by CRISPR/Cas9-mediated complete ablation of MHC expression, JCI Insight. 2019; 4(20):e129739. https://doi.org/10.1172/jci.insight.129739.
Nankivell, B. J. & Kuypers, D. R. Diagnosis and prevention of chronic kidney allograft loss. The Lancet (2011). doi:10.1016/S0140-6736(11)60699-5
Pallet, N. et al. Rapamycin inhibits human renal epithelial cell proliferation: effect on cyclin D3 mRNA expression and stability. Kidney Int. 67, 2422-33 (2005).
Picascia A, Infante T, Napoli C. Luminex and antibody detection in kidney transplantation. Clin Exp Nephrol. 2012 June; 16(3):373-81. doi: 10.1007/s10157-012-0635-1. Epub 2012 May 3. PMID: 22552384
Reindl-Schwaighofer, R. et al. Contribution of non-HLA incompatibility between donor and recipient to kidney allograft survival: genome-wide analysis in a prospective cohort. Lancet (London, England) 393, 910-917 (2019).
Satchell, S. C. et al. Conditionally immortalized human glomerular endothelial cells expressing fenestrations in response to VEGF. Kidney Int. 69, 1633-1640 (2006).
See, S. B. et al. Post-Transplant Natural Antibodies Associate with Kidney Allograft Injury and Reduced Long-Term Survival. J. Am. Soc. Nephrol. 29, 1761 LP-1770 (2018).
Senev, A. et al. Histological picture of antibody-mediated rejection without donor-specific anti-HLA antibodies: Clinical presentation and implications for outcome. Am. J. Transplant. 19, 763-780 (2019).
Serreze, D. V, Leiter, E. H., Christianson, G. J., Greiner, D. & Roopenian, D. C. Major histocompatibility complex class I-deficient NOD-B2mnull mice are diabetes and insulitis resistant. Diabetes 43, 505-9 (1994).
Schinstock, C. A. et al. Banff survey on antibody-mediated rejection clinical practices in kidney transplantation: Diagnostic misinterpretation has potential therapeutic implications. Am. J. Transplant. 19, 123-131 (2019).
Steers, N. J. et al. Genomic Mismatch at LIMS1 Locus and Kidney Allograft Rejection. N. Engl. J. Med. 380, 1918-1928 (2019).
Stoeckius, M. et al. Simultaneous epitope and transcriptome measurement in single cells. Nat. Methods 14, 865-868 (2017).
Tait Brian D., Detection of HLA Antibodies in Organ Transplant Recipients —Triumphs and Challenges of the Solid Phase Bead Assay, Frontiers in Immunology, 2016, 7: 570, doi: 10.3389/fimmu.2016.00570
U.S. Pat. No. 5,049,386—N-ω,(ω-1)-dialkyloxy)- and N-(ω,(ω-1)-dialkenyloxy)Alk-1-YL-N,N,N-tetrasubstituted ammonium lipids and uses therefor
U.S. Pat. No. 4,946,787—N-(ω,(ω-1)-dialkyloxy)- and N-(ω,(ω-1)-dialkenyloxy)-alk-1-yl-N,N,N-tetrasubstituted ammonium lipids and uses therefor
U.S. Pat. No. 4,897,355—N[ω,(ω-1)-dialkyloxy]- and N-[ω,(ω-1)-dialkenyloxy]-alk-1-yl-N,N,N-tetrasubstituted ammonium lipids and uses therefor
WO 2020/144366 A1—In Vitro Method For Determining The Likelihood Of Occurrence Of An Acute Microvascular Rejection (Amvr) Against A Renal Allograft In An Individual

1. A method for determining the likelihood of occurrence of a non-HLA antibody mediated rejection against a renal allograft in an individual in need thereof and administering a treatment against a non-HLA antibody mediated rejection against a renal allograft in said individual in need thereof, the method comprising at least the steps of:
i) carrying out an in vitro method comprising at least the steps of:
a) incubating at least one engineered human glomerular endothelial cell with a blood sample of said individual, said blood sample being presumed to contain non-HLA antibodies, said human glomerular endothelial cell being engineered to suppress the expression of HLA I and HLA II,
b) obtaining a ratio of a first quantification of a detectable signal obtained from said antibodies of said blood sample bound to said engineered human glomerular endothelial cell over a second quantification of a detectable signal from a human glomerular endothelial cell engineered to suppress the expression of HLA I and HLA II, in absence of non-HLA antibodies,
c) comparing the ratio obtained at step b) with a predetermined reference value, wherein said predetermined reference value being a ratio of a first quantification of a detectable signal obtained from antibodies bound to a human engineered glomerular endothelial cell, said human glomerular endothelial cell being engineered to suppress the expression of HLA I and HLA II, said antibodies being from a blood sample of an individual known to contain non-HLA antibodies over a second quantification of a detectable signal obtained from a human engineered glomerular endothelial cell, said human glomerular endothelial cell being engineered to suppress the expression of HLA I and HLA II, and
d) wherein a ratio obtained at step b) greater than said predetermined reference value indicates that said individual is at risk of a non-HLA antibody mediated rejection against a renal allograft,
and
ii) administering to said individual a treatment for preventing and/or reducing a non-HLA antibody mediated rejection against a renal allograft if said patient is determined to be at risk of a non-HLA antibody mediated rejection against a renal allograft.

2. (canceled)

3. The method of claim 1 wherein in the in vitro method the suppression in the expression is mediated by a CRISPR/Cas9 gene disruption of a gene encoding B2M and/or a gene encoding CIITA.

4. The method of claim 1 wherein in the in vitro method the disruption is in exon 1 and/or in exon 2 of the gene encoding B2M, and/or the disruption is in exon 2 and/or in exon 3 of a gene encoding CIITA.

5. The method of claim 1 wherein in the in vitro method the cell is a CiGEnCΔHLA, in particular the cell line deposited at the Institut Pasteur under the reference CNCM I-5707.

6. The method of claim 1 wherein in the in vitro method the individual is selected from the group consisting of (i) a candidate individual for a renal allograft and (ii) a recipient of a renal allograft.

7. The method of claim 1 wherein in the in vitro method said quantification is obtained with a labelled anti-human immunoglobulin antibody, or a fragment thereof.

8.-11. (canceled)

12. A kit for determining the likelihood of occurrence of a non-HLA antibody mediated rejection against a renal allograft in an individual in need thereof, the kit comprising:
(i) at least one engineered human glomerular endothelial cell, said human glomerular endothelial cell being engineered to suppress the expression of HLA I and HLA II,
(ii) at least one means to detect and quantify antibodies bound on said human glomerular endothelial cell, and
(iii) an instruction to compare a ratio of a first quantification of a detectable signal obtained from antibodies bound to an engineered human glomerular endothelial cell, said human glomerular endothelial cell being engineered to suppress the expression of HLA I and HLA II, said antibodies being from a blood sample of an individual presumed to contain non-HLA antibodies, over a second quantification of a detectable signal obtained from engineered human glomerular endothelial cells, said human glomerular endothelial cell being engineered to suppress the expression of HLA I and HLA II, in absence of non-HLA antibodies, with a predetermined reference value, said predetermined reference value being a ratio of a first quantification of a detectable signal obtained from antibodies bound to a human engineered glomerular endothelial cell, said human glomerular endothelial cell being engineered to suppress the expression of HLA I and HLA II, said antibodies being from a blood sample of an individual known to contain non-HLA antibodies over a second quantification of a detectable signal obtained from an engineered glomerular endothelial cell, said human glomerular endothelial cell being engineered to suppress the expression of HLA I and HLA II, in absence of non-HLA antibodies.

13. The kit according to claim 12, wherein the engineered human glomerular endothelial cell is a CiGEnCΔHLA, in particular the cell line deposited at the Institut Pasteur under the reference CNCM I-5707.

14. The kit according to claim 12, wherein the means to detect and quantify antibodies bound on said human glomerular endothelial cell is a labeled anti-human immunoglobulin antibody, or a fragment thereof.

15-16. (canceled)

17. The method of claim 1, wherein said treatment is selected among immunosuppressant drugs, plasma exchanges; immuno-adsorptions; intravenous immune globulins; or drugs targeting antibodies, B lymphocytes or plasma cells depleting agents.

18. The method of claim 1, wherein said ratio is a ratio of geometric means of fluorescence intensity.

19. The method of claim 1, further comprising a step of determining the likelihood of occurrence of an anti-HLA antibody mediated rejection against a renal allograft.

20. A method for diagnosing an occurrence of a non-HLA antibody mediated rejection against a renal allograft in a recipient of a renal allograft in need thereof and administering a treatment against a non-HLA antibody mediated rejection against a renal allograft in said individual in need thereof, the method comprising at least the steps of:
i) carrying out the in vitro method comprising at least the steps of
a) incubating at least one engineered human glomerular endothelial cell comprising a reduction in expression of a human leukocyte antigen (HLA) with a blood sample of said individual, said blood sample being presumed to contain non-HLA antibodies,
b) obtaining a quantification of a predetermined signal of non-HLA antibodies bound to said engineered human glomerular endothelial cell,
c) comparing the quantification obtained at step b) with a predetermined reference value, wherein the predetermined reference value is a ratio of a first quantification of a predetermined signal obtained from antibodies bound to an engineered glomerular endothelial cell comprising a reduction in expression of a human leukocyte antigen (HLA), said antibodies being from a blood sample of an individual known to contain non-HLA antibodies over a second quantification of a predetermined signal obtained from an engineered glomerular endothelial cell comprising a reduction in expression of a human leukocyte antigen (HLA), in absence of non-HLA antibodies, and
d) wherein a quantification obtained at step b) greater than the predetermined reference value indicates that said recipient is undergoing a non-HLA antibody mediated rejection against a renal allograft,
and
ii) administering to said individual a treatment for preventing and/or reducing a non-HLA antibody mediated rejection against a renal allograft if said patient is determined to be at risk of a non-HLA antibody mediated rejection against a renal allograft.
